(19) United States
(12) Patent Application Publication
(10) Pub. No.: US 2026/0210973 A1
(43) Pub. Date: Jul. 23, 2026

(54) METHODS FOR PROGNOSIS AND MONITORING PULMONARY HYPERTENSION

(71) Applicants: INSTITUT NATIONAL DE LA SANTÉ ET DE LA RECHERCHE MÉDICALE, Paris (FR); UNIVERSITÉ PARIS-SACLAY, Gif-sur-Yvette (FR); ASSISTANCE PUBLIQUE-HÔPITAUX DE PARIS (APHP), Paris (FR)

(72) Inventors: Christophe GUIGNABERT, Le Kremlin Bicêtre (FR); Laurent SAVALE, Le Kremlin Bicêtre (FR); Ly ieng TU, Le Kremlin Bicêtre (FR); Marc HUMBERT, Le Kremlin Bicêtre (FR); Athénaïs BOUCLY, Le Kremlin Bicêtre (FR); Olivier SITBON, Le Kremlin Bicêtre (FR)

(21) Appl. No.: 19/137,780

(22) PCT Filed: Dec. 20, 2023
(86) PCT No.: PCT/EP2023/086809

§ 371 Date: Jun. 11, 2025

(30) Foreign Application Priority Data
Dec. 21, 2022 (EP) 22306977.4

(51) Int. Cl. G01N 33/68

(52) U.S. Cl. CPC G01N 33/6893; G01N 2800/12; G01N 2800/321; G01N 2800/50; G01N 2800/52

(57) ABSTRACT

In the present invention, in Pulmonary Arterial Hypertension (PAH) patient's blood samples (EFFORT cohort), inventors results indicate that activin-Smad2/3 signaling is overactive, as reflected by the overabundance of inhibin-βA, inhibin-βB, activin type-I and type-II receptors, and phospho-Smad2/3 in PAH vascular endothelial and smooth muscle cells and by the fact that elevated levels of activin A and follistatin-like 3 (FSTL3) level in serum predict adverse outcome. With an independent external PAH cohort, inventors confirmed that both activin A and FSTL3 are prognostic biomarkers in PAH. Thereafter, this approach allows to identify a BMP/TGF ligands combinations that represent a reliable biomarker of PAH severity and/or mortality, validated in a second independent cohorts of PAH patient (Imperial College of London, UK study) 2-biomarker panel composed of activin A and follistatin-like 3 (FSTL3) that was independently associated with prognosis both at the time of PAH diagnosis and at the first follow-up after PAH therapy initiation. More specifically present invention relates to methods for prognosis and/or monitoring of the severe form of Pulmonary Hypertension (PH) and PAH through comparison of specific markers combinations in PH or PAH patient.

### FIELD OF THE INVENTION

The present invention relates to methods and kits for prognostic and monitoring Pulmonary Hypertension (PH) and in particular Pulmonary Arterial Hypertension (PAH). More specifically present invention relates to methods for prognosis of Pulmonary Hypertension (PH) and in particular Pulmonary Arterial Hypertension (PAH) through detection of a specific BMP/TGFβ (bone morphogenetic protein/transforming growth factor-beta/) ligand signature in a patient.

### BACKGROUND OF THE INVENTION

Pulmonary arterial hypertension (PAH) refers to a rare and severe cardiopulmonary disorder in which occlusive remodeling of the small peripheral lung vasculature is largely responsible for the rise in pulmonary vascular resistance (PVR) and mean pulmonary artery pressure (mPAP), resulting in right heart failure and ultimately death without effective intervention^{1,2}. Patient survival of advanced PAH remains poor with a 5-year survival rate about 60% 3-5, and lung transplantation remains a suitable option for eligible patients who are refractory to the currently approved PAH medications and continue to have progressive clinical deterioration⁶.

Growing preclinical and clinical evidence indicates a detrimental role played by an enhanced Smad2/3-mediated transcriptional response, often accompanied by impaired bone morphogenetic protein (BMP)-Smad1/5/8 signal transduction⁷, in PAH⁸⁻¹¹. Although the exact mechanisms behind these alterations are unknown, activin A—the dimer of inhibin-βA subunit encoded by INHBA—is suspected of having a role in both the Smad2/3-pathway overactivation and the progression of obstructive vascular remodeling in PAH¹¹⁻¹⁴. Elevated levels of activin A and of its functional antagonist follistatin were detected in small patient cohorts and an association between activin A levels and mortality has been noted^{11,14} In addition, mice with an endothelial cell (EC)-specific overexpression of inhibin-βA (VEcad-INHBA-Tg mice) spontaneously develop PH and pulmonary vascular remodeling, whereas mice with an EC-specific conditional deletion of inhibin-βA (INHBA-ECKO mice) are more resistant to the development of PH induced by chronic hypoxia¹³. Furthermore, the recent Phase 2 randomized controlled trial PULSAR (A Study of Sotatercept for the Treatment of Pulmonary Arterial Hypertension) met its primary endpoint of a reduction in PVR, indicating that sotatercept, a novel, first-in-class fusion protein composed of the extracellular domain of the human activin receptor type IIA (ActRIIA) fused to the Fc domain of human IgG1, has the potential to serve as an add-on therapy in PAH patients receiving background therapy^{12,15} More recently, the Phase 3 randomized controlled trial STELLAR demonstrated significant improvement in exercise capacity and key secondary outcome measures compared to placebo when added to background therapy (NCT04576988). Other phase 3 trials are now underway in newly diagnosed Intermediate- and High-Risk PAH (HYPERION, NCT04811092) and in high risk patients despite maximal background therapy (ZENITH, NCT04896008). Although different TGF-β family ligands bind with high affinity to ActRIIA, including among others activin A (inhibin-βA-inhibin-βA), activin B (inhibin-βB-inhibin-βB), GDF8, and GDF11, the precise mechanism of action of sotatercept in PAH still remains unknown.

Previous studies have suggested a role of the activin-Smad2/3 signaling in pathological remodeling of the vascular wall in PAH¹¹⁻¹⁴, atherosclerosis and other cardiovascular diseases¹⁶⁻¹⁸, but a comprehensive analysis of gene expression profiling of the different key members of the activin signaling system (FIG. 1) in the serum and lung tissues of PAH patients is currently lacking. In addition, the prognostic value of circulating activin signaling members at baseline and during follow-up as biomarkers has never been studied. Therefore, the main objective of this translational invention was to analyze the prognostic impact of serum levels of activin ligands and their inhibitors at baseline and during follow-up in a French prospective PH cohort of patients with idiopathic, heritable, or anorexigen-associated PAH and to determine whether the PAH pulmonary vascular lesions are associated with differential expression patterns of the activin signaling members.

The inventors therefore set up a prognostic and monitoring method of Pulmonary Hypertension (PH) and in particular Pulmonary Arterial Hypertension (PAH) that allows direct access to the BMP/TGFβ signaling dysfunctions of the patient that is critical for risk stratification and assessment of disease progression.

### SUMMARY OF THE INVENTION

A first object of the present invention relates to an in vitro method for assessing a subject's risk of having or developing a severe form of Pulmonary Hypertension (PH), comprising the steps of i) determining in a sample obtained from the subject the level of follistatin-like 3 (FSTL3) alone or optionally with the level of activin A ii) comparing the level determined in step i) with a reference value for each marker and iii) concluding
when the level of follistatin-like 3 (FSTL3) alone or optionally with the level of activin A, determined at step i) are higher than the reference value for each marker, is predictive of a high risk of having or developing a severe form of Pulmonary Hypertension (PH) and/or
when the level of follistatin-like 3 (FSTL3) alone or optionally with the level of activin A, determined at step i) are lower than the reference value for each marker is predictive of a low risk of having or developing a severe form of Pulmonary Hypertension (PH).

In a particular embodiment, the Pulmonary Hypertension (PH) is Pulmonary Arterial Hypertension (PAH).

In one embodiment, the Pulmonary Hypertension (PH) is Chronic thromboembolic pulmonary hypertension (CTEPH).

An additional object of the invention relates to an in vitro method for monitoring Pulmonary Hypertension (PH) disease comprising the steps of i) determining the level of follistatin-like 3 (FSTL3) alone or optionally with the level of activin A in a sample obtained from the subject at a first specific time of the disease, ii) determining the level of follistatin-like 3 (FSTL3) alone or optionally with the level of activin A in a sample obtained from the subject at a second specific time of the disease, iii) comparing the levels determined at step i) with the levels determined at step ii) and iv) concluding that the disease has evolved in worse manner when the level of follistatin-like 3 (FSTL3) alone or optionally with the level of activin A, determined at step ii) is higher than the levels determined at step i).

An additional object of the invention relates to an in vitro method for monitoring the treatment of Pulmonary Hypertension (PH) comprising the steps of i) determining the level of follistatin-like 3 (FSTL3) alone or optionally with the level of activin A in a sample obtained from the subject before the treatment, ii) determining the level of follistatin-like 3 (FSTL3) alone or optionally with the level of activin A in a sample obtained from the subject after the treatment”, iii) comparing the levels determined at step i) with the levels determined at step ii) and iv) concluding that the treatment is efficient when the level of follistatin-like 3 (FSTL3 alone or optionally with the level of activin A determined at step ii) is lower than the level determined at step i).

In a particular embodiment, the Pulmonary Hypertension (PH) is Pulmonary Arterial Hypertension (PAH).

In one embodiment, the Pulmonary Hypertension (PH) is Chronic thromboembolic pulmonary hypertension (CTEPH).

In a particular embodiment regarding the method for prognosis and monitoring (the disease or the treatment) the Pulmonary Hypertension (PH) is Pulmonary Arterial Hypertension (PAH).

In a particular embodiment regarding the method for prognosis and monitoring (the disease or the treatment) the Pulmonary Arterial Hypertension (PAH) is selected from the list consisting of: idiopathic Pulmonary Arterial Hypertension (iPAH), heritable Pulmonary Arterial Hypertension (heritable PAH) and drug- and toxin-induced (anorexigen) Pulmonary Arterial Hypertension (PAH).

### DETAILED DESCRIPTION OF THE INVENTION

In the present invention, in PAH patient's blood and lungs samples, inventors' results indicate that activin-Smad2/3 signaling is overactive, as reflected by the overabundance of inhibin-βA, inhibin-βB, activin type-I and type-II receptors, and phospho-Smad2/3 in PAH vascular endothelial and smooth muscle cells and by the fact that elevated levels of activin A and follistatin-like 3 (FSTL3) levels in serum predict adverse outcome. With an independent external PAH cohort, inventors confirmed that both activin A and FSTL3 are prognostic biomarkers in PAH. Furthermore, inventors show that overactivation of activin-Smad2/3 signaling in PAH is accompanied by alterations in the abundance of several activin antagonistic regulators within remodeled pulmonary arterial walls, including of the inhibin α-subunit, follistatin, FSTL3, and Cripto.

Briefly, risk stratification and assessment of disease progression in patients with pulmonary arterial hypertension (PAH) are challenged by the lack of accurate disease-specific and prognostic biomarkers. To date, B-type natriuretic peptide (BNP) and/or its N-terminal fragment (NT-proBNP) is the only marker for right ventricular dysfunction used in clinical practice in association with echocardiographic and invasive hemodynamic variables to predict outcome in patients with PAH. This study was designed to identify an easily measurable biomarker panel in the serum of well-phenotyped PAH patients with idiopathic, heritable, or drug-induced PAH at baseline and first follow-up. Among all biomarkers studied, inventors identified a 2-biomarker panel composed of activin A and follistatin-like 3 (FSTL3) and that these markers levels were independently associated with prognosis both at the time of PAH diagnosis and at the first follow-up after PAH therapy initiation. Furthermore, the prognostic value of the two biomarkers remains statistically significant after adjustment to usual non-invasive variables. The results were validated in a fully independent external validation cohort (Cohort from Imperial College of London, UK study). The monitoring of follistatin-like 3 (FSTL3) alone or optionally with activin A levels in serum should be considered in the management and treatment of patients with PAH to objectively guide therapeutic options.

Together these data suggest that point of care determining the signature of BMP/TGF ligands might help distinguish patients most likely to benefit or not from at least one approved PAH-specific therapies, including among other an endothelin receptor antagonists (ERAs) therapy, phosphodiesterase type-5 inhibitors therapy, and prostacyclin analogs therapy or lung surgery therapy. These results thus set-up the basis for the development of a rapid functional specific test for PAH and also improved personalized patient management through BMP/TGFβ ligand profiling.

### Prognostic Methods According to the Invention

The present invention relates to an in vitro method for assessing a subject's risk of having or developing a severe form of Pulmonary Hypertension (PH), comprising the steps of i) determining in a sample obtained from the subject the level of follistatin-like 3 (FSTL3) alone or optionally with the level of activin A ii) comparing the level determined in step i) with a reference value for each marker and iii) concluding
when the level of follistatin-like 3 (FSTL3) alone or optionally with the level of activin A, determined at step i) are higher than the reference value for each marker, is predictive of a high risk of having or developing a severe form of Pulmonary Hypertension (PH) and/or
when the level of follistatin-like 3 (FSTL3) alone or optionally with the level activin A, determined at step i) are lower than the reference value for each marker is predictive of a low risk of having or developing a severe form of Pulmonary Hypertension (PH).

In another term the present invention relates to an in vitro prognosis method of having or developing a severe form of Pulmonary Hypertension (PH) comprising the steps of i) determining in a sample obtained from the subject the level of follistatin-like 3 (FSTL3 alone or optionally with the level activin A ii) comparing the level determined in step i) with a reference value for each marker and iii) concluding
when the level of follistatin-like 3 (FSTL3 alone or optionally with the level of activin A, determined at step i) are higher than the reference value for each marker, is predictive of a high risk of having or developing a severe form of Pulmonary Hypertension (PH) and/or
when the level of follistatin-like 3 (FSTL3) alone or optionally with the level of activin A, determined at step i) are lower than the reference value for each marker is predictive of a low risk of having or developing a severe form of Pulmonary Hypertension (PH).

In a particular embodiment the Pulmonary Hypertension (PH) is Pulmonary Arterial Hypertension (PAH).

In a particular embodiment the Pulmonary Arterial Hypertension (PAH) is selected from the list consisting of: idiopathic Pulmonary Arterial Hypertension (iPAH), heritable Pulmonary Arterial Hypertension (heritable PAH) and drug- and toxin-induced (anorexigen) Pulmonary Arterial Hypertension (toxin-induced PAH).

The term “prognosis” is a medical term for predicting the likely or expected development of a disease. Prognostic scoring is also used for disease outcome predictions.

In the context of the present invention the “prognosis” is associated with changes in the levels of key BMP/TGF-β ligands comprising the level of follistatin-like 3 (FSTL3) alone or optionally with the level of Activin A markers which in turn may be a risk for developing a severe form of Pulmonary Hypertension (PH) and in particular of Pulmonary Arterial Hypertension (PAH)

The term “subject” as used herein refers to a mammalian, such as a rodent (e.g., a mouse or a rat), a feline, a canine or a primate. In a preferred embodiment, said subject is a human subject. The subject according to the invention can be a healthy subject or a subject suffering from a given disease such as Pulmonary Hypertension (PH) and in particular Pulmonary Arterial Hypertension (PAH).

In particular embodiments, the subject of the present invention suffers from Pulmonary Hypertension (PH) and in particular Pulmonary Arterial Hypertension (PAH) and/or have been previously diagnosed with PH and in particular PAH.

In particular embodiments, a plurality of BMP/TGF ligand signatures (“BMP/TGF-β Biomarker”: follistatin-like 3 (FSTL3) alone or optionally with Activin A) may be used in the methods of prognostic/prognostic of survival/classification/monitoring/treatment response/of the invention. In other words, the methods of the invention may comprise steps of: detecting in the biological sample the level of 1, 2, BMP/TGF-β biomarkers present in the biological sample; and detecting any biomarker of the invention.

In preferred embodiments, the methods of prognostic/prognostic of survival/classification/monitoring/treatment response are performed using the 2 different BMP/TGF-β biomarkers including follistatin-like 3 (FSTL3) and activin A.

In particular embodiment, the methods of prognostic/prognostic of survival/classification/monitoring/treatment response are performed using 1 biomarker (follistatin-like 3 (FSTL3)) or the 2 key BMP/TGF-β biomarkers (follistatin-like 3 (FSTL3) and activin A).

As used herein, the term “Pulmonary Hypertension” or “PH” and the term “Pulmonary Arterial Hypertension” or “PAH” has its general meaning in the art and refers to Pulmonary hypertension (PH) which defines a group of clinical conditions presenting with abnormal elevation in the pulmonary circulation pressure. Thus, a normal mean pulmonary artery pressure (mPAP) at rest is 14±3.3 mm Hg, and a PH is commonly defined as an increase of mPAP>20 mm Hg at rest, as assessed by right heart catheterization. The PH diseases are classified into five classes: class 1 to class 5 (Simonneau et al., Eur Respir J. 2019 Jan. 24; 53(1):1801913 and Humbert et al., Eur Respir J. 2022 Aug. 30; 2200879). In particular, pulmonary hypertension diseases include pulmonary arterial hypertension (group 1), PH due to left heart disease (group 2), PH due to lung diseases and/or hypoxia (group 3), chronic thromboembolic pulmonary hypertension (group 4), and other PH conditions with unclear multifactorial mechanisms (group 5) (Simonneau et al., Eur Respir J. 2019 Jan. 24; 53(1):1801913 and Humbert et al., Eur Respir J. 2022 Aug. 30; 2200879).

Among pulmonary hypertension diseases, the pulmonary arterial hypertension is a devastating pulmonary vascular disease-causing breathlessness, loss of exercise capacity and ultimately death. As recently, pointed by the inventors, this disease is characterized by a chronic increase in pulmonary artery pressure (above 20 mmHg), caused by an important remodeling of small pulmonary vessels associated to inflammation, leading to progressive vessel occlusion, ultimately leading to right ventricular failure and death (Humbert et al., Eur Respir J. 2019 Jan. 24; 53(1):1801887).

There is unfortunately no cure of PAH. The current PAH therapies are essentially focused on decreasing pulmonary vascular resistance by stimulating pulmonary vasodilation (prostacyclin analogues, phosphodiesterase type 5 inhibitors, and endothelin receptor antagonists) (Humbert et al., N. Engl. J. Med. 2004, O'Callaghan D S, et al. Nat. Rev. Cardiol., 2014). These agents have some anti-remodeling properties, but there is no current anti-remodeling strategy approved for PAH. In spite of these treatments targeting endothelial cell dysfunction that are now available to improve quality of life and survival, in most patients the outcome is very poor. Median survival of PAH (that was 2.8 years in the 1980's) remains inferior to 5 years and refractory cases are candidates for heart-lung transplantation, a major surgery with current limitations due to shortage of organ donors and severe long-term complications (5-year survival is only 50%). Some hemodynamic and clinical effects of the tyrosine kinase inhibitor imatinib have also been reported in severe PAH, but at the expense of severe side effects.

According to WHO (World Health Organisation) classification there are 5 groups of PH (pulmonary hypertension), where Group I (pulmonary arterial hypertension) is further subdivided into Group I′ and Group I″ classes (Simonneau G, et al. (2009). Journal of the American College of Cardiology. 54 (1 Suppl): S43-54 and Simonneau G, et al. (2013). Journal of the American College of Cardiology. 62 (25 Suppl): D34-41). The most recent WHO classification system (with adaptations from the more recent ESC/ERS guidelines) can be summarized as follows (Humbert et al., Eur Respir J. 2022 Aug. 30; 2200879):

### GROUP 1: Pulmonary Arterial Hypertension (PAH)

1.1 Idiopathic
   1.1.1 Non-responders at vasoreactivity testing
   1.1.2 Acute responders at vasoreactivity testing
1.2 Heritable
1.3 Associated with drugs and toxins
1.4 Associated with:
   1.4.1 Connective tissue disease
   1.4.2 HIV (human immunodeficiency virus) infection
   1.4.3 Portal hypertension
   1.4.4 Congenital heart disease
   1.4.5 Schistosomiasis
1.5 PAH with features of venous/capillary (pulmonary veno-occlusive disease (PVOD), pulmonary capillary hemangiomatosis (PCH)) involvement
1.6 Persistent PH of the newborn
   GROUP 2: PH Associated with Left Heart Disease
2.1 Heart failure:
   2.1.1 with preserved ejection fraction
   2.1.2 with reduced or mildly reduced ejection fraction
2.2 Valvular heart disease
2.3 Congenital/acquired cardiovascular conditions leading to post-capillary PH
   GROUP 3: PH Associated with Lung Diseases and/or Hypoxia
3.1 Obstructive lung disease or emphysema
3.2 Restrictive lung disease
3.3 Lung disease with mixed restrictive/obstructive pattern
3.4 Hypoventilation syndromes
3.5 Hypoxia without lung disease (e.g., high altitude)
3.6 Developmental lung disorders
   GROUP 4: PH Associated with Pulmonary Artery Obstructions
4.1 Chronic thrombo-embolic PH
4.2 Other pulmonary artery obstructions
   GROUP 5: PH with Unclear and/or Multifactorial Mechanisms
5.1 Haematological disorders
5.2 Systemic disorders
5.3 Metabolic disorders
5.4 Chronic renal failure with or without haemodialysis
5.5 Pulmonary tumour thrombotic microangiopathy
5.6 Fibrosing mediastinitis

A “severe form of Pulmonary Hypertension (PH)” or “severe form of Pulmonary Arterial Hypertension (PAH)”, refers to the progression of the disease associated with high mortality rate related to the remodeling of precapillary pulmonary arteries, leading to right heart failure. Patient with severe form of PH or PAH are mostly current therapy refractory cases and are candidates for heart-lung transplantation. According to the 2022 European Society of Cardiology (ESC)/European Respiratory Society (ERS) PH guidelines (2022 ESC/ERS Guidelines for the diagnosis and treatment of pulmonary hypertension; Humbert et al., Eur Respir J. 2022 Aug. 30; 2200879), patients are classified as high risk when the estimated 1-year mortality exceeds 10%. Utilizing risk stratification tools or scores (i.e. REVEAL score ≥10) or ESC/ERS criteria) may be particularly useful to denote high-risk individuals (Boucly A, et al. Eur Respir J. 2017; 50. Boucly A, et al. Eur Respir J. 2022; 59. Hoeper M M, et al. Eur Respir J. 2022; 60).

In particular embodiments, the Pulmonary Arterial Hypertension (PAH) is idiopathic Pulmonary Arterial Hypertension (iPAH) or heritable Pulmonary Arterial Hypertension (heritable PAH) or drug- and toxin-induced (anorexigen) induced Pulmonary Arterial Hypertension (toxin-induced PAH).

As used herein, the term “sample” or “biological sample” as used herein refers to any biological sample of a subject and can include, by way of example and not limitation, bodily fluids and/or tissue extracts such as homogenates or solubilized tissue obtained from a subject. Tissue extracts are obtained routinely from tissue biopsy. In a particular embodiment regarding the prognostic method of the PAH according to the invention, the biological sample is a body fluid sample (such blood, serum or plasma) or tissue biopsy of said subject.

In preferred embodiments, the fluid sample is a blood sample. In one embodiment, the blood sample to be used in the methods according to the invention is a whole blood sample, a serum sample, or a plasma sample. In a preferred embodiment, the blood sample is a whole blood sample obtained from a subject (e.g., an individual for which it is interesting to determine whether a population of serum biomarkers can be identified).

As used herein, the term “follistatin-like 3” or “(FSTL)-3” also known as “FLRG” or “FSRP” has its general meaning in the art refers to a glycoprotein that in humans is encoded by the FSTL3 gene (gene ID 10272)/UniProtKB 095633). FSTL3, i.e., Follistatin-like 3 is a secreted glycoprotein of the follistatin-module-protein family that is physiologically released by adipose tissue, reproductive, glands, liver, heart, and especially placenta. Recently, significant FSTL3 overexpression was also found in some malignant tumors (Lung cancer: Gao L, et al Onco Targets Ther (2020) 13:2725-38, 45, 46; thyroid cancer: Panagiotou G, et al J Clin Endocrinol Metab (2021) 106: e2137-50) and may also serves as a Biomarker of Extracellular Matrix Remodeling in Colorectal Cancer (Chao Y. et al Front Immunol. 2021 Jul. 16; 12:717505. Follistatin (Uniprot P19883) is an autocrine glycoprotein that is expressed in nearly all tissues of higher animals (Tortoriello D V et al. (2001). Endocrinology. 142 (8): 3426-3434). Its primary function is the binding and bioneutralization of members of the TGF-β superfamily, with a particular focus on activin, a paracrine hormone.

As used herein, the term “Activin A” has its general meaning in the art and refers to a protein complex dimer composed of two identical (inhibin) beta subunits (Activin A=inhibin-βA-inhibin-βA). Inhibin-βA (or INHBA) subunit in humans is encoded by the INHBA gene (gene ID 3624)/UniProtKB P08476). Inhibin is also a protein complex dimer wherein the first component is a beta subunit similar or identical to the beta subunit in activin. However, in contrast to activin, the second component of the inhibin dimer is a more distantly-related alpha subunit (see Burger H G, Igarashi M (1988). The Journal of Clinical Endocrinology and Metabolism. 66 (4): 885-6). Activin, inhibin and a number of other structurally related proteins such as anti-Müllerian hormone, bone morphogenetic protein, and growth differentiation factor belong to the TGF-β protein superfamily (Kingsley D M (1994). “Genes & Development. 8 (2): 133-46; Guignabert and Humbert. Eur Respir J. 2021 Feb. 4; 57 (2): 2002341). The activin and inhibin protein complexes are both dimeric in structure, and, in each complex, the two monomers are linked to one another by a single disulfide bond (Ying S Y (1987). Proceedings of the Society for Experimental Biology and Medicine. 186 (3): 253-64). In addition, both complexes are derived from the same family of related genes and proteins but differ in their subunit composition. For example activin A (inhibin-βA-inhibin-βA), activin B (inhibin-βB-inhibin-βB), Inhibin A (inhibin-α-inhibin-βA), inhibin B (inhibin-α-inhibin-βB)

Activin and inhibin are two closely related protein complexes that have almost directly opposite biological effects. Identified in 1986, (Vale W, et al. (1986). Nature. 321 (6072): 776-9) activin enhances FSH biosynthesis and secretion, and participates in the regulation of the menstrual cycle. Many other functions have been found to be exerted by activin, including roles in cell proliferation, differentiation, apoptosis (Chen Y G, et al. (2006). Experimental Biology and Medicine. 231 (5): 534-44) metabolism, homeostasis, immune response, wound repair (Sulyok S, et al. (2004). Molecular and Cellular Endocrinology. 225 (1-2): 127-32) and endocrine function. Conversely, inhibin downregulates FSH synthesis and inhibits FSH secretion (van Zonneveld P, et al. (2003). Human Reproduction. 18 (3): 495-501).

The level of the markers of the invention may be determined by using standard electrophoretic and immunodiagnostic techniques, including immunoassays such as competition, direct reaction such as immunohistochemistry, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

Standard methods for detecting the level of specific biomarker such as: follistatin-like 3 (FSTL3) and activin A are well known in the art. Typically, the step consisting of detecting the marker may consist in using at least one differential binding partner directed against the marker.

For example, the serum cytokine level (β-follistatin-like 3 (FSTL3) and activin A can be determined using specific ELISA KIT such as for FSTL3 (AL-152, AnshLabs, TX, USA and for Activin A (DAC00B, Biotechne, Boston, USA).

As used herein, the term “binding partner directed against the marker” refers to any molecule (natural or not) that is able to bind the surface marker with high affinity. The binding partners may be antibodies that may be polyclonal or monoclonal, preferably monoclonal antibodies. In another embodiment, the binding partners may be a set of aptamers.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally; the human B-cell hybridoma technique; and the EBV-hybridoma technique.

The binding partners of the invention such as antibodies or aptamers may be labelled with a detectable molecule or substance, such as preferentially a fluorescent molecule, or a radioactive molecule or any others labels known in the art. Labels are known in the art that generally provide (either directly or indirectly) a signal.

As used herein, the term “labelled”, with regard to the antibody or aptamer, is intended to encompass direct labelling of the antibody or aptamer by coupling (i.e., physically linking) a detectable substance, such as a fluorophore [e.g. fluorescein isothiocyanate (FITC) or phycoerythrin (PE) or Indocyanine (Cy5)]) or radioactive molecule or a non-radioactive heavy metals isotopes to the antibody or aptamer, as well as indirect labelling of the probe or antibody by reactivity with a detectable substance. An antibody or aptamer of the invention may be labelled with a radioactive molecule by any method known in the art. More particularly, the antibodies are already conjugated to a fluorophore (e.g., FITC-conjugated and/or PE-conjugated).

The aforementioned assays may involve the binding of the binding partners (i.e., antibodies or aptamers) to a solid support. The solid surface could a microtitration plate coated with the binding partner for the surface marker. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled.

Such methods comprise contacting a biological sample obtained from the subject to be tested under conditions allowing detection of follistatin-like 3 (FSTL3) and Activin A markers. Once the sample from the subject is prepared, the level of biomarkers (“key TGF-β/BMP ligands signature”: follistatin-like 3 (FSTL3) and Activin A) may be measured by any known method in the art.

Typically, the high of PH-associated biomarkers (“TGF-β/BMP ligand signature”: (follistatin-like 3 (FSTL3) and Activin A) is intended by comparison to a control reference value.

Said reference control values may be determined in regard to the level of biomarker present in blood samples taken from one or more healthy subject(s) or to the cell surface biomarker in a control population.

In one embodiment, the method according to the present invention comprises the step of comparing said level of key TGF-β/BMP ligands-associated biomarkers, namely “key TGF-β/BMP ligand signature” (follistatin-like 3 (FSTL3) and activin A) to a control reference value for each marker wherein a high level of follistatin-like 3 (FSTL3) alone or with the level of Activin A marker compared to said respective control reference value (for each marker) is predictive of a high risk of having a severe form of Pulmonary Hypertension (PH) and/or Pulmonary Arterial Hypertension (PAH) and a low level of follistatin-like 3 (FSTL3) alone or with the level of Activin A marker compared to said control reference value (for each marker) is predictive of a low risk of having or developing a severe form of Pulmonary Hypertension (PH) and/or Pulmonary Arterial Hypertension (PAH).

The control reference value may depend on various parameters such as the method used to measure the level TGF-β/BMP ligands-associated biomarker (“Biomarker”: follistatin-like 3 (FSTL3) and Activin A) or the gender and the age of the subject.

Typically regarding the reference value using “TGF-β/BMP ligand signature” (follistatin-like 3 (FSTL3) and Activin A), as indicated in the Experimental section (see FIGS. 3 and 6), for the levels of TGF-β/BMP ligands using immunoassay approach identify and quantify TGF-β/BMP ligands (follistatin-like 3 (FSTL3) and Activin A) wherein the blood levels of TGF-β/BMP ligands (follistatin-like 3 (FSTL3) and Activin A) is superior to respectively 16.6 ng/ml, and 393 μg/ml is predictive of having or a high risk of having or developing a severe form of Pulmonary Arterial Hypertension (PAH). Conversely wherein the levels of TGF-β/BMP ligands (follistatin-like 3 (FSTL3) and Activin A) is inferior to respectively 16.6 ng/ml, and 393 μg/ml, is predictive of not having or at a low risk of having a severe form of Pulmonary Arterial Hypertension (PAH).

Control reference values are easily determinable by the one skilled in the art, by using the same techniques as for determining the level of cell surface biomarker or cell death in blood samples previously collected from the patient under testing.

A “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the levels of “Biomarker PH” follistatin-like 3 (FSTL3) and Activin A) with a defined threshold value for each marker. In one embodiment of the present invention, the threshold value is derived from the inflammatory cytokine levels (or ratio, or score) determined in a blood sample derived from one or more subjects who are responders (to the method according to the invention). In one embodiment of the present invention, the threshold value may also be derived from inflammatory cytokine level (or ratio, or score) determined in a blood sample derived from one or more subjects or who are non-responders (i.e., asymptotic subject). Furthermore, retrospective measurement of the PAH markers levels (or ratio, or scores) in properly banked historical subject samples may be used in establishing these threshold values.

Reference values are easily determinable by the one skilled in the art, by using the same techniques as for determining the level of “Biomarker PAH” follistatin-like 3 (FSTL3) and Activin A) in fluids samples previously collected from the patient under testing.

“Risk” in the context of the present invention, relates to the probability that an event will occur over a specific time period, as in the conversion to a severe form of Pulmonary Hypertension (PH) and/or Pulmonary Arterial Hypertension (PAH), and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no conversion. Alternative continuous measures, which may be assessed in the context of the present invention, include time to severe form of Pulmonary Hypertension (PH) and/or Pulmonary Arterial Hypertension (PAH) conversion risk reduction ratios.

“Risk evaluation” or “evaluation of risk” in the context of the present invention encompasses making a prediction of the probability, odds, or likelihood that an event or disease state may occur, the rate of occurrence of the event or conversion from one disease state to another, i.e., from a normal condition or asymptomatic form of PAH or symphonic form of PAH to a severe form of Pulmonary Hypertension (PH) and/or Pulmonary Arterial Hypertension (PAH) condition or to one at risk of developing a severe form of Pulmonary Hypertension (PH) and/or Pulmonary Arterial Hypertension (PAH). Risk evaluation can also comprise prediction of future clinical parameters, traditional laboratory risk factor values, or other indices of severe form of Pulmonary Hypertension (PH) and/or Pulmonary Arterial Hypertension (PAH), such as cellular population determination in peripheral tissues, in serum or other fluid, either in absolute or relative terms in reference to a previously measured population. The methods of the present invention may be used to make continuous or categorical measurements of the risk of conversion to a severe form of Pulmonary Hypertension (PH) and/or Pulmonary Arterial Hypertension (PAH), thus prognosing and defining the risk spectrum of a category of subjects defined as being at risk for a severe form of Pulmonary Hypertension (PH) and/or Pulmonary Arterial Hypertension (PAH). In the categorical scenario, the invention can be used to discriminate between normal and other subject cohorts at higher risk for severe form of Pulmonary Hypertension (PH) and/or Pulmonary Arterial Hypertension (PAH).

### Prognostic of Survival Methods of the Invention

As demonstrated by the inventors, distinct profiles of TGF-β/BMP ligands are observed in association with PH and/or PAH severity and are differentially predictive of mortality of PH and/or PAH patient. These results warrant new classification of PH and/or PAH patients based on cytokine profiling regarding the need or not of need for instance of lung surgery.

Accordingly, another object of the invention relates to an in vitro method for assessing a PH patient's risk of having a poor prognostic of survival, comprising the steps of i) determining in a sample obtained from the subject the level of follistatin-like 3 (FSTL3) alone or optionally with the level of Activin A markers ii) comparing the level determined in step i) with a reference value for each marker and iii) concluding
when the level of follistatin-like 3 (FSTL3) alone or optionally with the level of Activin A markers, determined at step i) are higher than the reference value for each marker, then said PH patient is at high risk of having a poor prognostic of survival; or
when the level of follistatin-like 3 (FSTL3) alone or optionally with the level of Activin A markers, determined at step i) are lower than the reference value for each marker are higher than the reference value then said PH patient is at high risk of having a good prognostic of survival.

In a particular embodiment the Pulmonary Hypertension (PH) is the Pulmonary Arterial Hypertension (PAH).

In a particular embodiment regarding the method for prognostic of survival the Pulmonary Arterial Hypertension (PAH) is selected from the list consisting of: idiopathic Pulmonary Arterial Hypertension (iPAH); heritable Pulmonary Arterial Hypertension (heritable PAH) and drug and toxin-induced (anorexigen) induced Pulmonary Arterial Hypertension (toxin-induced PAH)

### Monitoring Methods and Management

After the identification of markers subsets that harbour an alteration in the TGF-β/BMP signature, inventors highlighted, that the two “TGF-β/BMP ligands/biomarkers”: follistatin-like 3 (FSTL3) and activin A were independently associated with prognosis both at the time of PAH diagnosis and at the first follow-up after PAH therapy initiation. High levels of follistatin-like 3 (FSTL3) and activin A were predictors of death or transplantation. Accordingly, inventors provided evidence that this cytokine subset may serve as a severity biomarker in PH and/or PAH for prognosis and monitoring purpose (pathology or treatment).

Accordingly, an additional object of the invention relates to an in vitro method for monitoring Pulmonary Hypertension (PH) disease comprising the steps of i) determining the level of follistatin-like 3 (FSTL3) alone or optionally with the level of activin A in a sample obtained from the subject at a first specific time of the disease, ii) determining the level of follistatin-like 3 (FSTL3) alone or optionally with the level activin A in a sample obtained from the subject at a second specific time of the disease, iii) comparing the levels determined at step i) with the levels determined at step ii) and iv) concluding that the disease has evolved in worse manner when the level of follistatin-like 3 (FSTL3) alone or optionally with the level of activin A, determined at step ii) is higher than the levels determined at step i).

An additional object of the invention relates to an in vitro method for monitoring the treatment of Pulmonary Hypertension (PH)) comprising the steps of i) determining the level of follistatin-like 3 (FSTL3) alone or optionally with the level of Activin A in a sample obtained from the subject before the treatment, ii) determining the level of follistatin-like 3 (FSTL3) alone or optionally with the level of Activin A in a sample obtained from the subject after the treatment”, iii) comparing the levels determined at step i) with the levels determined at step ii) and iv) concluding that the treatment is efficient when the level of follistatin-like 3 (FSTL3) alone or optionally with the level of Activin A determined at step ii) is lower than the level determined at step i).

In a particular embodiment the Pulmonary Hypertension (PH) is the Pulmonary Arterial Hypertension (PAH).

In one embodiment, the Pulmonary Hypertension (PH) is Chronic thromboembolic pulmonary hypertension (CTEPH).

In a particular embodiment regarding the method for prognosis and monitoring (the disease or the treatment) the Pulmonary Arterial Hypertension (PAH is selected from the list consisting of: idiopathic Pulmonary Arterial Hypertension (iPAH); heritable Pulmonary Arterial Hypertension (heritable PAH) and drug and toxin-induced (anorexigen) induced Pulmonary Arterial Hypertension (toxin-induced PAH).

The decrease or increase (depending of biomarkers) can be e.g., at least 5%, or at least 10%, or at least 20%, more preferably at least 50% even more preferably at least 100%.

Chronic thromboembolic pulmonary hypertension (CTEPH) is a rare and progressive pulmonary vascular disease characterized by elevated pulmonary arterial pressure resulting from pulmonary vascular obstruction combined with microvasculopathy, often arising post-pulmonary embolism. Treatment involves a multimodal approach, including pulmonary endarterectomy (PEA), balloon pulmonary angioplasty (BPA), and medical intervention with specific pulmonary vasodilators based on lesion extension and localization. Despite PEA being the gold standard treatment for proximal lesions, tools to quantify microvasculopathy extension and residual PH post-PEA are lacking.

In one embodiment, the method according to the invention is suitable for predicting microvasculopathy extension and residual PH in patients eligible for PEA.

In one embodiment, the method according to the invention is suitable for predicting the survival of patients eligible for PEA. Therapeutic Method of a specific population

As mentioned, endothelin receptor antagonists, phosphodiesterase type 5 (PDE-5) inhibitors, and prostacyclin analogues are the current approved treatments for Pulmonary Hypertension (PH) and especially for Pulmonary Arterial Hypertension (PAH). Even if these drugs have markedly improved overall quality of life, exercise capacity, and long-term outcomes²⁻⁶, the 5-year survival rate for patients suffering from PH or PAH remains low (around 60%)⁷⁻⁹, and lung transplantation remains an important treatment option for eligible patients with severe PAH if medical treatment fails¹⁰. Multiparametric risk stratification at the time of PH or PAH diagnosis and at follow-up provides useful information for the choice of first-line therapy and for subsequent treatment escalation.

The present study shows that cytokine profiling may have clinical implications for improved personalized treatment. For example, the present invention allows to identify a novel panel of two cytokines (follistatin-like 3 (FSTL3) and Activin A) in serum independently associated with prognosis at both baseline and at the first follow-up after PAH therapy initiation. Subsequent analysis revealed that the prognostic value of this 3-biomarker panel was more powerful for predicting PAH survival than usual clinical and hemodynamic variables. Therefore, it is proposed that serum FSTL3 and Activin A levels should be considered in the management and treatment of patients with PAH during follow-up to objectively identify patients with a high risk of death to adapt treatment (treatment escalation and/or lung transplantation).

Accordingly, the invention also relates to a method for treating Pulmonary Hypertension (PH) with endothelin receptor antagonist and/or phosphodiesterase type 5 (PDE-5) inhibitor and/or and prostacyclin analogues in a subject wherein the level of follistatin-like 3 (FSTL3) alone or optionally with the level of Activin A markers obtained from said subject, have been detected, by one of the methods of the invention.

Thus, the invention also relates to a method for guiding personalized therapy of Pulmonary Hypertension (PH), with either endothelin receptor antagonist treatment and/or phosphodiesterase type 5 (PDE-5) inhibitor and/or prostacyclin derivative according to the cytokine profile of the subject.

In the context of the invention, the term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies.

The term “endothelin receptor antagonists” or “ERA” means a class of a drug that blocks endothelin receptors.

### Three Main Kinds of ERAs Exist:

selective ETA receptor antagonists (ambrisentan, atrasentan, BQ-123, zibotentan), which affect endothelin A receptors.
dual antagonists (bosentan, macitentan, tezosentan), which affect both endothelin A and B receptors.
selective ETB receptor antagonists (BQ-788 and A192621) which affect endothelin B receptors are used in research but have not yet reached the clinical trial stage.

Ambrisentan and bosentan are mainly used for the treatment of pulmonary arterial hypertension, while atrasentan is an experimental anti-cancer drug.

Edonentan is an endothelin receptor antagonist drug.

In a particular embodiment, endothelin receptor antagonists according to the invention can be ambrisentan and bosentan

The term “phosphodiesterase type 5 inhibitors” or “PDE5 inhibitor” is a vasodilating drug which works by blocking the degradative action of cGMP-specific phosphodiesterase type 5 (PDE5) on cyclic GMP in the smooth muscle cells lining the blood vessels supplying various tissues. For instance, these drugs dilate the corpora cavernosa of the penis, are used in the treatment of erectile dysfunction (ED). Accordingly, Phosphodiesterase-5 (PDE5) inhibitors such as sildenafil (Viagra), tadalafil (Cialis), and vardenafil (Levitra) are clinically indicated for the treatment of erectile dysfunction. Because PDE5 is also present in the smooth muscle of the walls of the arterioles within the lungs, two PDE5 inhibitors, sildenafil and tadalafil, are FDA/EMD-approved for the treatment of pulmonary hypertension while tadalafil (Levitra) is also licensed for the treatment of benign prostatic hyperplasia. As of 2019, the wider cardiovascular benefits of PDE5 inhibitors are being appreciated (Tzoumas N, et al. (2019). British Journal of Pharmacology. 177 (24): 5467-5488).

In a particular embodiment, PDE5 inhibitor according to the invention can be: sildenafil and tadalafil.

The term “Prostacyclin” (also called prostaglandin 12 or PGI2) is a prostaglandin member of the eicosanoid family of lipid molecules. It inhibits platelet activation and is also an effective vasodilator. When used as a drug, it is also known as epoprostenol and the terms are sometimes used interchangeably (Kermode J, et al. (1991). “British Heart Journal. 66 (2): 175-178).

Prostacyclin is commonly considered the most effective treatment for PAH. Epoprostenol (synthetic prostacyclin) is given via continuous infusion that requires a semi-permanent central venous catheter. This delivery system can cause sepsis and thrombosis. Prostacyclin is unstable, and therefore has to be kept on ice during administration. Other Prostacyclin derivatives have therefore been developed. Treprostinil can be given intravenously or subcutaneously, but the subcutaneous form can be very painful. An increased risk of sepsis with intravenous Remodulin has been reported by the CDC. Iloprost is also used in Europe intravenously and has a longer half-life. Iloprost was the only inhaled form of prostacyclin approved for use in the US and Europe, until the inhaled form of treprostinil was approved by the FDA in July 2009

In a particular embodiment, Prostacyclin derivatives according to the invention can be: Epoprostenol, Treprostinil Remodulin and Iloprost.

Another object of the present invention is a method of treating Pulmonary Hypertension (PH) in a subject comprising the steps of:
a) providing a blood sample from a subject,
b) detecting the level of follistatin-like 3 (FSTL3) alone or optionally with the level of Activin A markers
c) comparing the level determined at stet b) with a reference value for each marker and
if the level of follistatin-like 3 (FSTL3) marker alone or optionally with the level of Activin A marker, determined at step i) are lower than the reference value for each marker then, treating the subject with endothelin receptor antagonists and/or phosphodiesterase type 5 (PDE-5) inhibitors and/or prostacyclin derivatives.

If the level of follistatin-like 3 (FSTL3) marker alone or optionally with the level of Activin marker, determined at step i) are higher than the reference value for each marker then treating the subject with treatment escalation of endothelin receptor antagonists and/or phosphodiesterase type 5 (PDE-5) inhibitors and/or and prostacyclin derivatives and/or lung transplantation.

In a particular embodiment the Pulmonary Hypertension (PH) is the Pulmonary Arterial Hypertension (PAH).

In particular embodiments, the Pulmonary Arterial Hypertension (PAH) is selected from the list consisting of: idiopathic Pulmonary Arterial Hypertension (iPAH), heritable Pulmonary Arterial Hypertension (heritable PAH) and drug- and toxin-induced (anorexigen) Pulmonary Arterial Hypertension (PAH).

The invention will be further illustrated by the following figures and examples. However, these examples and figures should not be interpreted in any way as limiting the scope of the present invention.

### FIGURES

FIG. 1: Levels of activin A, activin B, the α-subunit of inhibin A and B proteins, and of the antagonists follistatin (FST) and FST like-3 (FSTL3) in the serum of patients with Pulmonary Arterial Hypertension (PAH) at diagnosis, first treatment follow-up, and in the serum of healthy subjects. ns: non-significant. * and # p-value <0.05.

FIG. 2: Kaplan-Meier survival curves according to levels of activin-A and FSTL3 at the diagnosis (A, B) and at the follow-up (C, D): Thresholds determined by ROC curves: 440 μg/mL for Activin-A and 16.2 ng/ml for FSTL3. Log rank p<0.001 for both analyses.

FIG. 3: A positive correlation between serum circulating activin-A and FTSL3 at baseline (r=0.622, p<0.001) and at follow-up (r=0.746, p<0.001) FIG. 4: Time dependent ROC curves of transplant-free survival of Activin A (A) and FSTL3 (B) at the diagnosis.

FIG. 5: Kaplan-Meier survival curves according to levels of activin-A and FSTL3 at diagnosis (A, B) and at follow-up (C, D) in the validation cohort. Thresholds determined by time-dependent ROC curves: 393 μg/mL for Activin-A and 16.6 ng/mL for FSTL3).

FIG. 6: Kaplan-Meier survival curves according to the number of low-risk status of activin A and FSTL3 at the diagnosis (A) and at the follow-up (B) in the French cohort.

FIG. 7: Comparison of circulating levels of Activin-A and FSTL-3 between CTEP patients and controls

### EXAMPLE

### Methods:

### Immunofluorescence and Confocal Microscopy

For the in situ studies, we used lung specimens obtained during lung transplantation in patients with PAH and during lobectomy or pneumonectomy for localized lung cancer in control subjects (Data not shown). Preoperative echocardiography was performed in these control patients to rule out pulmonary hypertension (PH). The lung specimens from the controls were collected at a distance from the tumor foci. The absence of tumoral infiltration was retrospectively established in all tissue sections by the histopathological analysis. This study was approved by the local ethics committee (CPP EST-III n°18.06.06, Le Kremlin-Bicêtre, France). All patients gave informed consent before the study.

Immunofluorescence staining of α-smooth muscle actin (α-SMA), ACTRIIA, ACTRIIB, ALK2, ALK4, ALK5, ALK7, BAMBI (BMP and activin membrane bound inhibitor), betaglycan (TGFβRIII), CD31, cripto, connective tissue growth factor (CTGF), follistatin, follistatin-like 3 (FTSL3 or FLRG), inhibin-α, inhibin-βA, inhibin-βB, plasminogen activator inhibitor (PAI)-1 (serpine), phospho-Smad2/3, and von Willebrand factor (vWF) were performed in human lung paraffin sections (Table 5). Briefly, 5-μm thick lung sections were deparaffinized and incubated in retrieval buffer. The sections were then saturated with blocking buffer and incubated overnight with specific antibodies, followed by corresponding secondary fluorescently labeled antibodies (Thermo Fisher Scientific). Nuclei were labeled using DAPI (Thermo Fisher Scientific). Slides were mounted using ProLong Gold antifade reagent (Thermo Fisher Scientific). The images were captured using an LSM700 confocal microscope (Zeiss) with ZEN software.

### Cohort Data Collection

Discovery cohort: This study was conducted in accordance with the Declaration of Helsinki and informed consent was obtained for each patient prior to their enrolment. This is an ancillary study from the EFORT (Evaluation of prognostic FactORs and Treatment goals in PAH) cohort (NCT 01185730). The ‘EFORT’ study is a prospective study to assess prognostic factors at both baseline and follow up in a French cohort of incident (i.e., newly diagnosed) patients with PAH. All incident patients entered in the French Registry between January 2011 and December 2013 with a diagnosis of idiopathic, heritable or anorexigen-induced PAH were enrolled in the EFORT study.

Assessments have been performed at baseline (i.e., time of PAH diagnosis), 3-4 months after treatment initiation or treatment change, and then once a year until a 3-year follow up for patients included into the study in the first two years (2011-2012) and until a 2-year follow up for patients included in the last year (2013). Serial assessments included New York Heart Association (NYHA) functional class (FC), non-encouraged six-minute walk test (6 MWT), right heart catheterization and biomarkers BNP (B-type natriuretic peptide) or NT-proBNP (N-terminal pro BNP). An ancillary study was performed in 80 patients with collection of serum samples at both baseline and first follow-up visit for cytokines measurements. These 80 patients constituted our study population.

Validation cohort: A London cohort of 129 PAH patients constituted our validation cohort collected as part of the Imperial College Prospective Study of Patients with Pulmonary Vascular Disease cohort (UK Research Ethics Committee approval EC Reference 17/LO/0563). A collection of serum samples was available in 125 patients at time of PAH diagnosis and in 37 patients during follow-up.

Healthy subjects: Peripheral blood from healthy volunteers was provided by the “Etablissement Français du Sang (EFS)” from Paris.

### Ella™ Microfluidic Platform and ELISA

Activin A (DAC00B, Biotechne, Boston, USA), activin B (AL-150, AnshLabs, TX, USA), FSTL3 (AL-152, AnshLabs, TX, USA), and inhibin-α subunit (AL-A34, AnshLabs, TX, USA) were measured using specific ELISA kits according to the manufacturer instructions. CXCL9, IL1-α, IL-6, follistatin, and GDF15 were measured using the SimplePlex Ella™ microfluidic platform (Protein Simple, CA, USA) according to the manufacturer instructions.

### Statistical Analyses

Data were collected from the web-based French PH Registry (PAHTool®; Inovultus Ltd. Santa Maria da Feira, Portugal). Statistical analysis was performed using SPSS Statistics version 26 (SPSS Inc., Chicago, IL). Continuous variables are expressed as the means±SD or medians (interquartile range, 25% to 75% [IQR]) according to the data distribution.

Serum levels of activin ligands and their inhibitors in PAH patients were compared with the serum of healthy controls (blood donors) by unpaired Student's t-test. Comparisons between levels of biomarkers at baseline and the first follow-up were performed by paired t test or nonparametric test according to the data distribution.

Univariable and multivariable forward stepwise Cox proportional hazards regression models were performed to determine the risk of event (death or transplantation) according to baseline and first follow-up visit variables. Variables with a p-value≤0.1 in the univariable analysis were eligible for entry into the multivariable models only if they were not highly correlated (absolute value of Spearman's p<0.6) with other variables and p>0.05 was the threshold for variable removal.

Variables identified in univariable analysis to be significantly associated with the prognosis at both baseline and follow-up were considered biomarkers of interest. For each of them, time-dependent ROC curves were performed to determine the best threshold by Youden's index.

The cutoff date was Dec. 31, 2020. Transplant-free survival was represented using the Kaplan-Meier method. To analyse the prognostic values of activin A and FSTL3 according to the threshold determined by the ROC analysis collected at baseline, we performed Kaplan Meier analyses with the date of baseline visit as a start point for survival analyses. Similarly, to analyse the prognostic values of Activin A and FSTL3 collected at first follow-up, we performed Kaplan Meier analyses with the date of follow-up visit as a start point for survival analyses.

Patients with idiopathic, heritable or anorexigen-induced PAH from the UK in whom blood samples were collected at the time of PAH diagnosis and during follow-up were used for external validation. In this validation cohort, survival analyses were performed using the Kaplan-Meier method.

### Results

### Elevated Serum Levels of Key Members of the Activin Signaling in PAH Patients

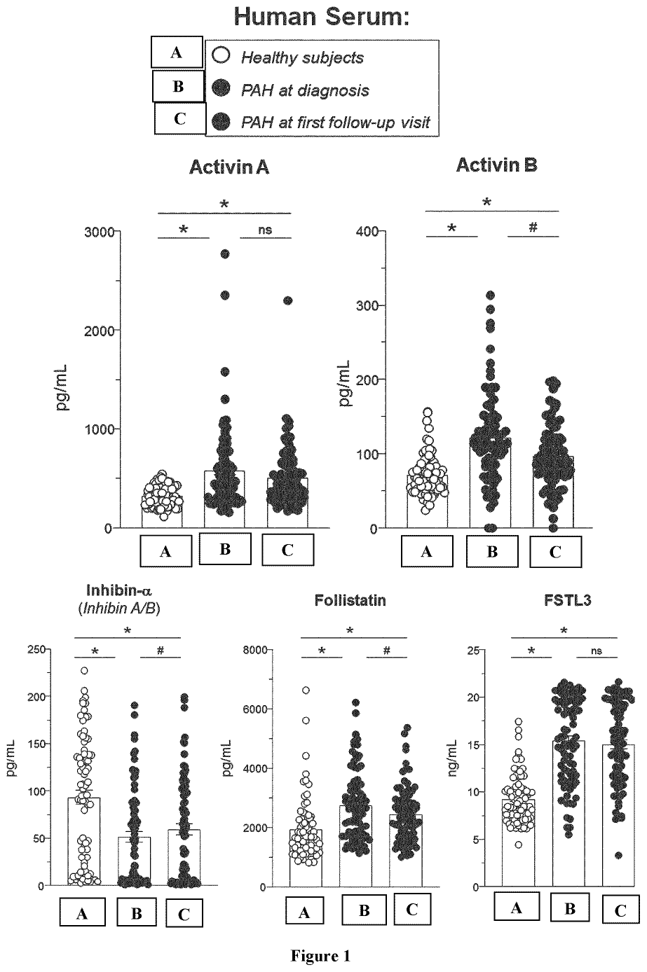

Circulating levels of activin A, activin B, of the α-subunit of inhibin A and B proteins, and of the two antagonists follistatin and FSTL3 were measured in incident patients with PAH (n=80) at baseline and during follow-up in the prospective French EFORT cohort of patients with idiopathic, heritable, or anorexigen-associated PAH and in healthy subjects (n=79) (FIG. 1 and Table 1).

A substantial increase in circulating activin A and activin B protein levels were observed in the serum of patients with newly diagnosed PAH and PAH-treated patients compared with healthy controls (FIG. 1). Furthermore, reduced activin B levels were found in PAH-treated patients relative to patients with newly diagnosed PAH (FIG. 1). Because inhibins share the β-subunits with activins and have the ability to inhibit activins by forming high affinity complexes with ACTRII and betaglycan, we also determined circulating levels of the inhibin α-subunit and found decreased levels in the serum of patients with newly diagnosed PAH and PAH-treated patients compared with healthy controls. Interestingly, some PAH patients exhibited very low levels of inhibin A/B (FIG. 1). Our blood analyses also revealed increases in follistatin or FSTL3 protein levels in PAH samples (FIG. 1). Interestingly, some PAH patients exhibited very high levels of follistatin and FSTL3. This increase in follistatin and FSTL3 might be partly explained by a feedback loop whereby follistatin is upregulated in response to activin A signals as described by Bartholin and colleagues (Bartholin et al., Oncogene. 2002; 21:2227-35).

Taken altogether these results indicate that PAH is associated with elevated serum levels of activin A, activin B, follistatin and FSTL3 in PAH patients at diagnosis and at first treatment follow-up, and with low levels of inhibin A and B.

### Elevated Levels of Activin A and of FSTL3 in Serum Predicts Adverse Outcome in PAH

Risk stratification and assessment of disease progression in PAH patients is challenged by the lack of accurate early diagnostic and prognostic biomarkers. Therefore, we explored the prognostic value of serum levels of activin ligands and their inhibitors at baseline and during follow-up in the prospective French EFORT cohort of patients with idiopathic, heritable, or anorexigen-associated PAH.

Between January 2011 and December 2013, 140 incident patients with idiopathic, heritable, or anorexigen-induced PAH were enrolled in the EFORT study 20. Among them, 80 patients with both a baseline and follow-up measurement of circulating biomarkers in addition to PAH evaluation, including NYHA-FC, 6-min WD, right heart catheterization (RHC) and NT-proBNP or BNP, were analysed. The patient characteristics are detailed in Table 1. First repeated clinical assessment after PAH therapies initiation was performed after a median (IQR) period of 4.6 (3.9-7.3) months following the first line therapy initiation.

After a median follow-up of 69 (IQR, 50-81) months, 21 patients had died and 5 underwent lung transplantation. In the overall cohort, comparison of baseline biomarkers of activin pathway between event-free survivors and non-survivors showed a significantly higher median value of activin A, follistatin, FSTL3 in non-survivors, while levels of activin-B and Inhibin-α were similar. Similarly, median values of activin A, follistatin, FSTL3 were higher in non-survivors at first follow-up (Table 6).

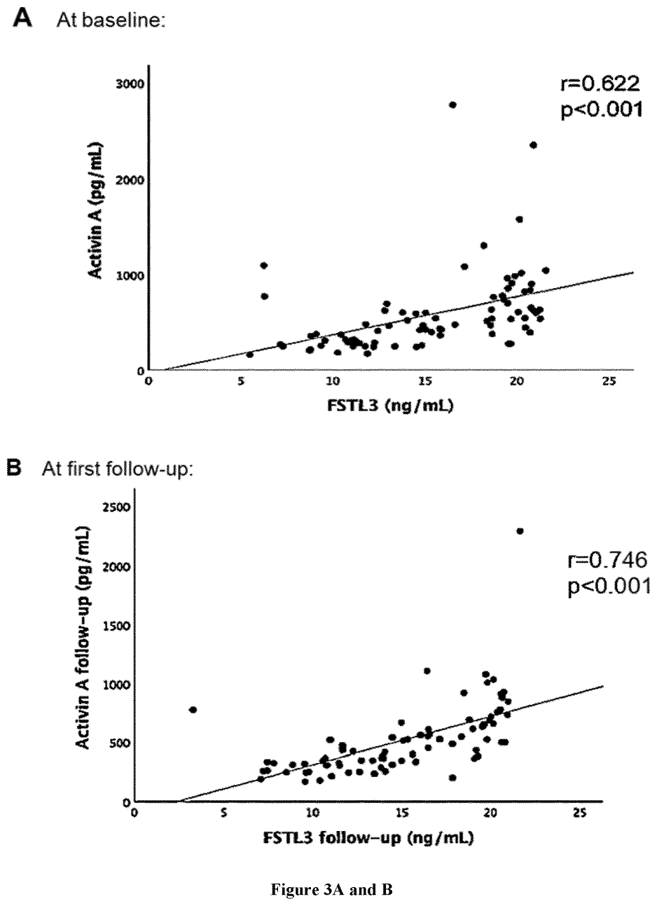

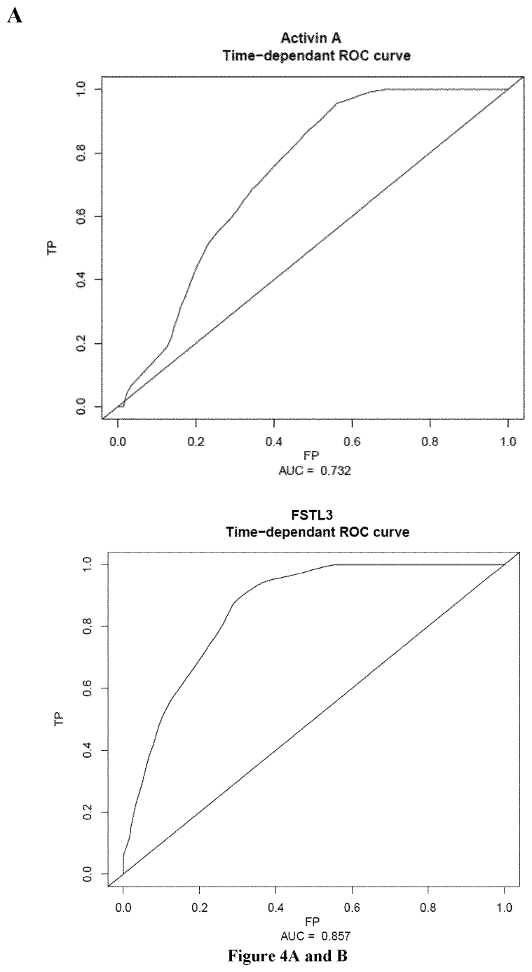

Univariable Cox proportional HR for biomarkers of activin pathway at baseline and follow-up, are presented in Table 2. Activin-A and FSTL3 were associated with transplant-free survival at both baseline and follow-up. Follistatin was associated with transplant-free survival at follow-up only. The relationship between elevated activin-A or elevated FSTL3 and transplant-free survival persisted in a model adjusted for age and sex at baseline and follow-up. A positive correlation between serum circulating activin-A and FTSL3 were found at baseline (r=0.622, p<0.001) and at follow-up (r=0.746, p<0.001) (FIG. 3).

For these two biomarkers, ROC curve analyses were performed to determine the best threshold of transplant-free survival at baseline: 440 μg/mL for activin-A and 16.2 ng/ml for FSTL3. The ROC curves from which the thresholds were identified are presented in FIG. 4. Comparison of clinical, hemodynamic and biological values between patients with high and low levels of Activin-A are detailed in Table 3. We also compared circulating levels of key pro-inflammatory cytokines known to be associated to PAH 21, 22 among patients with high and low levels of activin-A. Interestingly, PAH patients with high levels of activin-A have higher RAP, lower 6 MWD, higher values of BNP, GDF-15, IL6, β-NGF, and CXCL9 (Table 3).

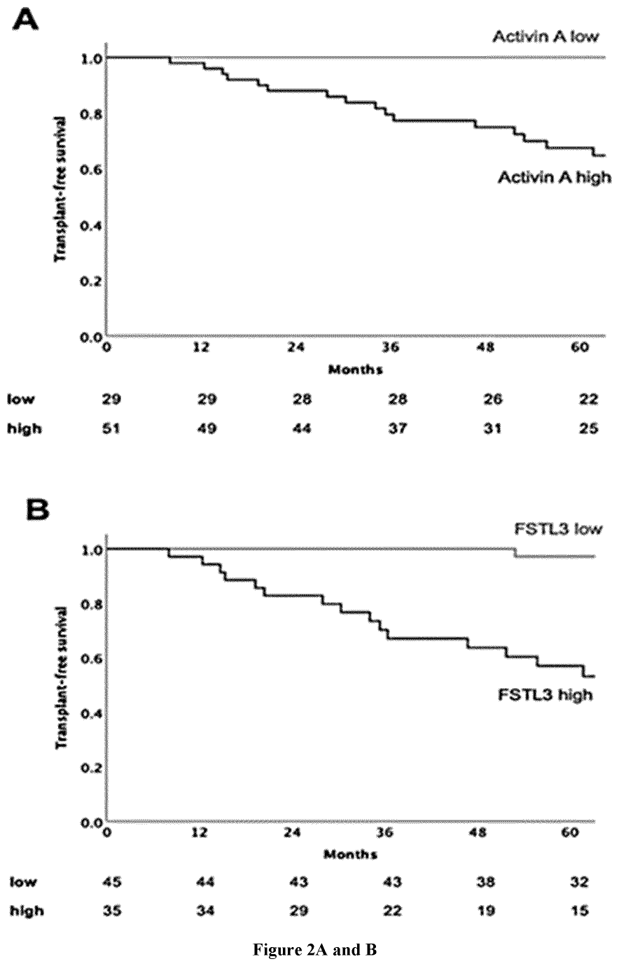

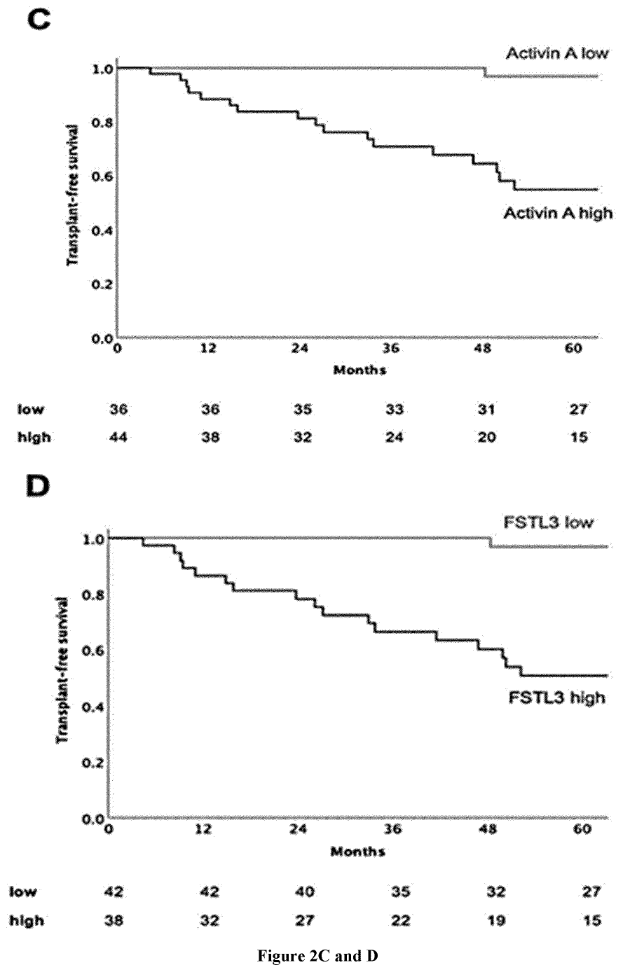

In univariable analysis, the hazard ratios of activin-A and FSTL3 expressed as dichotomous variables (according to thresholds previously determined) at diagnosis were 10.39 (3.07-35.12) (p<0.001) and 9.55 (3.26-28.01) (p<0.001) respectively. At first follow-up, the hazard ratios of activin-A and FSTL3 were 10.570 (4.193-26.643) (p<0.001) and 7.937 (3.298-19.102) (p<0.001) respectively. Kaplan-Meier survival curves according to thresholds of activin-A and FSTL3 are presented in FIG. 2. The prognostic value of activin-A (model A) and FSTL3 (model B) remain significant when adjusted with other non-invasive biomarkers currently used to perform risk assessment in PAH (NYHA-FC, 6 MWD and NT-proBNP) (Table 4).

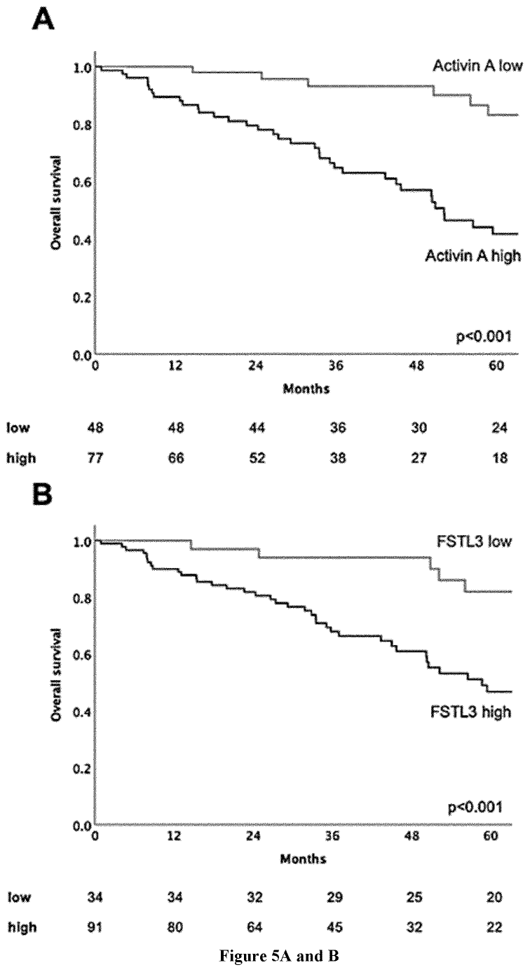

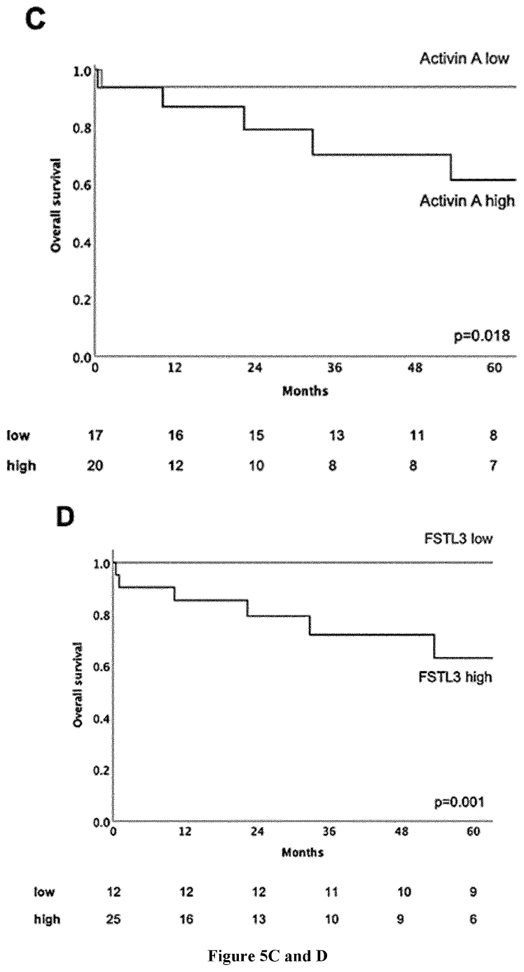

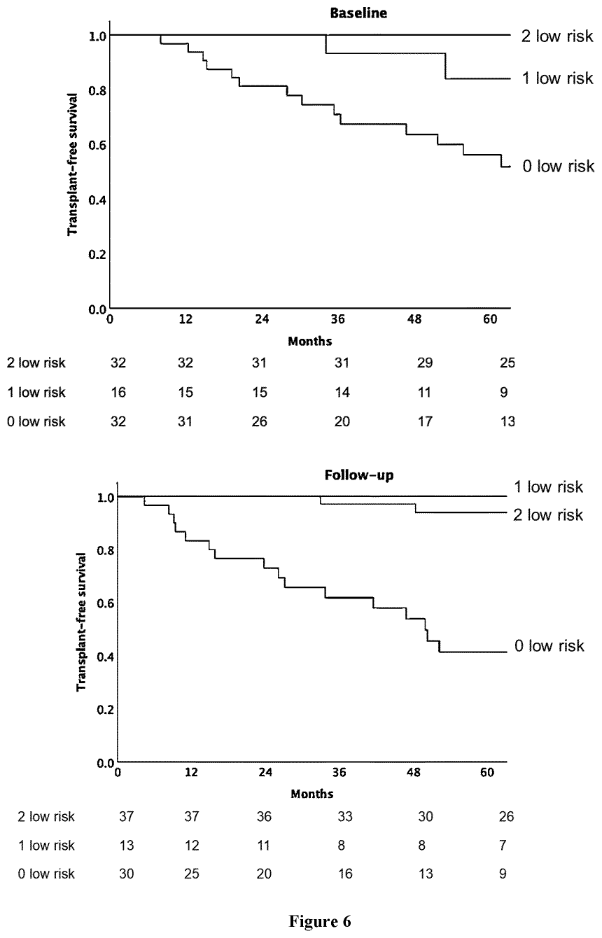

These findings were validated in an independent external PAH cohort comprised of 125 patients (69% female, mean age 59±17 years, 91% idiopathic PAH, 9% heritable PAH). Characteristics of the validation cohort are detailed in the Table 7. After a median follow-up of 59±30 months, 56 patients had died. A collection of serum samples was available in 125 patients at the time of PAH diagnosis and in 37 patients during follow-up. Kaplan-Meier survival analysis in this cohort according to the status of activin-A and FSTL3 confirmed the results previously observed in the French cohort (FIG. 5 and FIG. 6).

### Pulmonary Vascular Cells in PAH Overexpress the Two Activin Subunits Inhibin-βA and -βB

To determine whether the obstructive pulmonary vascular remodeling in PAH is associated with changes in the expression of activin ligands, confocal microscopic analyses and triple labeling of either α-, βA-, and βB-subunits with von Willebrand factor (vWF) and alpha-smooth muscle actin (α-SMA) were used to investigate the activin expression patterns in lung specimens from 4 patients with PAH and 4 control subjects (Supp. Table 3). We found strong staining for βA- and βB-subunits (Data not shown) a long with a weak staining of α-subunit in paraffin-embedded lungs of patients with PAH when compared with controls (Data not shown). More specifically, we found increased inhibin-βA immunogenicity within the endothelium of distal pulmonary arteries from patients with PAH as compared with weak staining of the endothelium of control specimens (fluorescence mean intensity (FMI): 12.7±1.1 versus 8.3±0.7, respectively, P>0.05) (Data not shown). Similar differential staining was observed for inhibin-βB (FMI: 43.4±19.3 versus 19.3±3.6, respectively, P>0.01), but we also noted a strong signal for inhibin-βB within the smooth muscle layer in PAH remodeled vessels relative to the smooth muscle of control specimens (FMI: 19.2±3.6 versus 8.0±0.7, respectively, P>0.05) (Data not shown). In addition, alveolar macrophages displayed strong immunogenicity for the two activin subunits inhibin-βA and inhibin-βB (Data not shown).

Taken altogether, these results support the thesis that both activin subunits inhibin-βA and inhibin-βB are overexpressed in the remodeled pulmonary arterial walls, with inhibin-βA expressed mainly by the dysfunctional pulmonary endothelium and inhibin-βB by both the vascular endothelial and smooth muscle layers.

### Increased Expression Patterns of Activin Receptors and Overactivation of Smad2/3 in PAH

Activin binding to ACTRIIA or ACTRIIB, is followed by recruitment, phosphorylation, and activation of the type I receptors to initiate signaling via intracellular Smad2/3 proteins. To study the expression of activin type-I, and type-II receptors on pulmonary vascular cells, subsequent studies were carried out using confocal microscopic analyses and triple labeling of either ACTRIIA, ACTRIIB, ALK2, ALK4, ALK5, and ALK7, with CD31 or von Willebrand factor (vWF) and alpha-smooth muscle actin (α-SMA) (Data not shown and Table 5) and lung specimens from 4 patients with PAH and 4 control subjects (Data not shown). In normal tissues, low expression of ACTRIIA and ACTRIIB, ALK2, and ALK7 was observed in pulmonary vessels. Furthermore, ALK4 and ALK5 were expressed by the endothelial and smooth muscle layers in control pulmonary vessels, respectively (Data not shown). In contrast, explanted PAH lung specimens showed strong staining for activin type-I and type-II receptors. More specifically, we found that ACTRIIB, ALK4, and ALK5 were highly expressed in the endothelium and smooth muscle of muscularized pulmonary arterioles in PAH and that ALK2 and ALK7 were highly expressed in the endothelium of muscularized pulmonary arterioles (FMI: 68.4±14.4 versus 19.8±2.9, P>0.05; FMI: 42.4±6.4 versus 1.7±0.7, P>0.001, respectively) (Data not shown). To assess the nuclear abundance of the phosphorylated form of Smad2/3 (phospho-Smad2/3) in pulmonary vascular cells in human PAH and control lungs, phospho-Smad2/3 immunofluorescent staining was next performed in human PAH and control lungs. Consistent with our previous findings, our confocal microscopic analyses demonstrated a nuclear accumulation of phospho-Smad2/3 within PAH remodeled arterial walls as reflected by a strong nuclear staining for phospho-Smad2/3 in both the vascular endothelial and smooth muscle layer relative to controls (FMI: 101.8±6.4 versus 16.7±3.9, P>0.001; FMI: 66.5±6.0 versus 21.2±1.3, P>0.001, respectively) (Data not shown).

To validate these observations, subsequent studies were carried out to study the expression patterns of the two known target genes downstream of SMAD2/3, named CTGF and PAI-1. Consistent with an overactivation of Smad2/3 in PAH, we found an increase in the staining for CTGF and PAI-1 in lungs of patients with PAH relative to controls (FMI: 21.7±1.7 versus 13.5±1.2, P>0.01; FMI: 31.1±4.1 versus 11.9±4.9, P>0.05, respectively) (Data not shown).

In conclusion, these findings obtained in explanted lungs from PAH patients revealed increased expression of activin type-I, and type-II receptors in vascular cells of remodeled pulmonary arterioles in PAH.

### Dysregulated Expression of Activin Regulators in Remodeled Pulmonary Vessels in PAH

Negative regulators of activin signals include the two activin-binding and neutralization proteins, follistatin and FTSL3^{23, 24}, and the cell membrane antagonistic co-receptors, betaglycan (TGFβRIII) and Cripto or BAMBI^{26,27}.

To determine whether the pulmonary vascular remodeling associated to PAH is associated with changes in follistatin or FSTL3 expression, immunofluorescent staining was next performed in human PAH and control lungs (Data not shown). Even if our confocal microscopic analyses revealed a small decrease in follistatin in the pulmonary endothelium and smooth muscle in PAH compared to control lungs (FMI: 8.2±0.5 versus 23.3±4.7, P>0.01; FMI: 30.6±9.4 versus 89.2±3.5, P>0.05, respectively), a substantial increase in FSTL3 proteins in PAH samples was observed (FMI: 69.4±2.8 versus 10.4±1.4, P>0.001; FMI: 51.0±6.3 versus 16.4±0.7, P>0.01, respectively). Although no difference was observed in the level of betaglycan, a low staining for the pseudo-receptor BAMBI that sequesters activin was also noted in both the vascular endothelial and smooth muscle layer in PAH relative to controls (FMI: 16.0±1.5 versus 25.6±3.0, P>0.05; FMI: 6.5±1.6 versus 26.0±3.6, P>0.01, respectively) (Data not shown). In addition, our confocal microscopic also revealed a strong expression of Cripto that blocks the recruitment of type-I receptors in vascular smooth muscle cells in PAH (FMI: 75.9±5.5 versus 16.9±3.7, P>0.001, respectively) (Data not shown).

Taken altogether, these findings suggest that activin-Smad2/3 signaling is overactive in PAH and accompanied by alterations in the abundance of several activin antagonistic regulators not only in the blood, but also within remodeled pulmonary arterial walls.

### DISCUSSION

A better understanding of dysregulated biological pathways involved in the exaggerated accumulation of vascular cells in the pulmonary arterial wall is needed to develop innovative PAH therapies. In this study, we present the first comprehensive analysis of protein expression profiling of the activin signaling system in the serum and explanted lungs of PAH patients. We provide evidence suggesting that activin-Smad2/3 signaling is overactive in human PAH and that elevated levels of activin A and FSTL3 levels in serum predict death or transplantation in a discovery cohort of PAH patients. We confirmed in an independent external PAH cohort that both activin-A and FSTL3 could serve as prognostic biomarkers in PAH. We also obtained evidence indicating that the two activin β-subunits, inhibin-βA and inhibin-βB, are abundant in both the vascular endothelial and smooth muscle layers of PAH remodeled pulmonary vessels, and that the abundance of inhibin A and B is reduced. Furthermore, we show that PAH vascular endothelial and smooth muscle cells exhibit high levels of phosphorylated Smad2/3 in their nuclei alongside overexpressed the activin type-I receptors ALK2, ALK4, ALK5, and ALK7 as well as the activin type-II receptor ACTRIIB. Increased activity of the activin-Smad2/3 signaling pathway in PAH is associated with alterations of several activin antagonistic regulators, namely follistatin, FSTL3, BAMBI and Cripto.

There are two earlier reports of elevated levels of activin A and follistatin in PAH patients^{10, 14}, but these studies are limited by the relatively small numbers of patients, a lack of independent validation cohorts, and absence of follow-up. The biological relevance of elevated levels of these proteins to PAH comes from their relationship to predict adverse outcomes in two independent patient cohorts. We found that serum levels of activin A and FSTL3 were outperformed traditional clinically employed measures in both our prospective and independent external validation cohort. The usual biomarkers incorporated into the current risk stratification tools are closely linked to the right ventricular dysfunction (BNP or NT-proBNP) and its systemic effect (eGFR)²⁷⁻³¹. Other biomarkers have been individually identified as prognostic factors, including growth and differentiation factor (GDF)-15²⁹, red cell distribution width (RDW)³⁰⁻³³, uric acid (UA)³⁴, creatinine³⁵, IL-6^{23-25,27,28} and angiopoietins³⁶, but none are used in daily practice. More recently, -omic studies allowed to enlarge the panel of biomarkers in PAH³³⁻³⁵. However, none of them have been currently identified as effective therapeutic targets. While the prognostic value of IL-6 as well as its implication in PAH pathophysiology have been demonstrated, anti-IL6R therapy failed to significantly improve pulmonary vascular resistance³⁶⁻³⁸. Similarly, FK506 increases BMPR-II expression in peripheral blood mononuclear cells but its long-term effect on PAH remains to be demonstrated³⁹. None of the licensed medications approved by the United States Food and Drug Administration (US FDA) to modulate abnormalities in three major pathobiological pathways for PAH (the nitric oxide, prostacyclin and endothelin pathways) are associated with a strong specific biomarker able to predict response to therapy and/or prognosis. Only elevated ET-1/ET-3 ratio has been identified as a potential prognostic factor of PAH but this observation has never been validated⁴⁰.

In contrast, activin-A is a therapeutic target and was identified in our study as a biomarker predictive of the survival of PAH patients. The prognostic value of activin-A remains strong and independent after adjustment for other non-invasive variables included in risk stratification tools (including 6 MWD, NYHA-FC and BNP or NT-proBNP). Of note, FSTL3 was also identified as a prognostic factor a both baseline and follow-up. However, a positive correlation between the abundance of activin A and FTSL3 was noted in our PAH serum samples that might be partly explained by a feedback loop upregulating follistatin in response to activin A signals¹⁹.

We next explored whether the obstructive pulmonary vascular remodeling in PAH was associated with changes in the expression of the different activin receptors and their inhibitory regulators in the serum and lung tissues of PAH patients. Our data indicate that explanted PAH lung specimens displayed strong staining for the activin type-II receptors ACTRIIA and ACTRIIB, as well as for the activin type-I receptors ALK2, ALK4, ALK5, and ALK7. In accordance, there was a significant increase in the staining of phosphorylated Smad2/3 levels in vascular endothelial and smooth muscle cells within remodeled pulmonary arterial walls in PAH lungs. Decreased circulating levels of inhibins newly diagnosed PAH compared with healthy controls and PAH-treated patients is of interest as these proteins have the ability to compete with activin and to block its action through an interaction with betaglycan and ACTRII. Interestingly, some PAH patients exhibited very low levels of inhibin A and B, but without substantial changes in betaglycan protein levels. The loss of the inhibins/betaglycan regulatory mechanism could, at least partly, contributes to the increase activin signaling in PAH. Because betaglycan is also known to bind TGF-β isoforms and other BMP/GDF ligands, we cannot exclude a role for this betaglycan overexpression in PA-SMC accumulation in PAH. In addition, we found a strong staining for the cell membrane antagonistic co-receptor betaglycan in the remodeled smooth muscle layer in explanted PAH lungs. Because betaglycan not only binds inhibin A but also TGF-β isoforms and other BMP/GDF ligands, we cannot exclude a role for this betaglycan overexpression in PA-SMC accumulation in PAH. Although no significant change was observed in the expression of BAMBI, we observed an overexpression of Cripto that is not only known to inhibit activin, but also to facilitate nodal signaling by forming receptor complexes with these ligands that are structurally and functionally similar 23. Therefore, further studies are needed to precise the contribution of these alterations in the activin-Smad2/3 signaling in PAH and to precise the underlying mechanisms.

The mechanism of action of the activin signaling system in the maintenance of vascular integrity is complex. We have already reported that inhibin-βA-specific endothelial activation exacerbates chronic hypoxia-induced PH, while its conditional endothelial knockout prevents the progression of PH in mice exposed to chronic hypoxia¹³. Mechanistically, we have previously reported that activin A triggers the internalization and subsequent degradation of BMPR-II, a phenomenon that can partly explain why enhanced activin-Smad2/3 signal transduction is often accompanied by the loss or impaired BMP-Smad1/5/8 response⁷. We were also able to demonstrate that primary cultures of pulmonary endothelial cells derived from PAH patients displayed higher inhibin-βA expression, and produced more activin A than pulmonary endothelial cells isolated from the lungs of normal control subjects¹³. Despite these findings, there is still gap in our knowledge on the underlying mechanisms behind activin overactivation in PAH and dysregulation of different negative regulators of activin signals. Given the established role of activin A as a proinflammatory and profibrotic mediator together with the recent observation that ACTRIIA-Fc can suppress inflammation in rodent models⁴⁰, studies of its involvement in PAH inflammatory component should be more closely examined. Of relevance, circulating levels of cytokines of interest were significantly increased in patients with levels of activin-A higher than the threshold determined by the ROC analysis in the EFORT cohort.

In summary, our study provides the first extensive analysis of protein expression profiling of the activin signaling system in human PAH. Although further experiments are required to identify the exact mechanisms underlying the activin overexpression in pulmonary vascular cells, we clearly demonstrated that the activin signaling system is overactive and that its serum level represents a prognostic biomarker that remains strong and independent after adjustment for all other variables included in risk stratification.

TABLE 1
Baseline characteristics for PAH patients in the EFORT cohort

| | Incident PAH patients from | |
|---|---|---|
| | EFORT cohort (n = 80) | |
| Female, n (%) | 57 | (71) |
| Age, years | 51 ± 19 | |
| PAH etiology, n (%) | | |
| Idiopathic | 53 | (66) |
| Heritable | 16 | (20) |
| Drug-induced PAH | 11 | (14) |
| NYHA functional class, n (%) | | |
| I-II/III/IV | 28 (35)/45 (56)/7 (9) | |
| 6MWD, m | 343 ± 143 | |
| Hemodynamics | | |
| RAP, mmHg | 9 ± 5 | |
| mPAP, mmHg | 51 ± 11 | |
| PAWP, mmHg | 9 ± 3 | |
| CO, L/min | 4.5 ± 1.2 | |
| CI, L/min/m² | 2.5 ± 0.6 | |
| PVR, WU | 10 ± 4 | |
| SvO₂, % | 63 ± 8 | |
| BNP, ng/L (n = 72) | 158 | (54-312) |
| NTproBNP, ng/L (n = 4) | 2349 | (106-4977) |
| First line treatment, n (%) | | |
| Calcium channel blocker | 5 | (6) |
| Oral monotherapy | 34 | (43) |
| Oral dual combination therapy | 33 | (41) |
| Triple combination therapy | 8 | (10) |

Abbreviations:
6MWD, 6-minute walk distance;
BNP; brain natriuretic peptide;
CI, cardiac index;
CO; cardiac output,
mPAP; mean pulmonary artery pressure;
NYHA; New York heart association;
PAH; pulmonary arterial hypertension;
PAWP; pulmonary artery wedge pressure;
PVR; pulmonary vascular resistance;
RAP; right atrial pressure;
Sv0₂; mixed venous oxygen saturation

TABLE 2

| | | |
|---|---|---|
| Hazard- | | P- |
| ratios | _{95%}CI | value |

A: at the diagnosis

| | | | |
|---|---|---|---|
| Activin A, pg/mL | 1.001 | 1.001-1.002 | <0.001 |
| Activin A, pg/mL (adjusted for age and | 1.001 | 1.000-1.001 | 0.037 |
| sex) | | | |
| Activin B, pg/mL | 1.000 | 0.992-1.007 | 0.923 |
| Follistatin, pg/mL | 1.000 | 1.000-1.001 | 0.069 |
| FSTL3, ng/mL | 1.353 | 1.173-1.560 | <0.001 |
| FSTL3, ng/mL (adjusted for age and | 1.263 | 1.049-1.520 | 0.014 |
| sex) | | | |
| Inhibin-α subunit, pg/mL | 1.001 | 0.992-1.010 | 0.861 |

B: at the first follow-up

| | | | |
|---|---|---|---|
| Activin A, pg/mL | 1.004 | 1.003-1.006 | <0.001 |
| Activin A, pg/mL (adjusted for age and | 1.003 | 1.001-1.005 | 0.001 |
| sex) | | | |
| Activin B, pg/mL | 0.997 | 0.987-1.007 | 0.547 |
| Follistatin, pg/mL | 1.001 | 1.000-1.001 | <0.001 |
| FSTL3, ng/mL | 1.365 | 1.185-1.573 | <0.001 |
| Follistatin, pg/mL (adjusted for age and | 1.280 | 1.088-1.506 | 0.003 |
| sex) | | | |
| Inhibin-α subunit, pg/mL | 0.998 | 0.990-1.006 | 0.622 |

Abbreviations: FSTL3: Follistatin-like 3. Biological values are expressed as continuous variables

TABLE 3
Comparison of clinical, hemodynamic, and biological parameters
between patients with high and low levels of circulating activin
A at baseline (Threshold determined by ROC analysis)

| | High level of activin A | | Low level of activin A | | P- |
|---|---|---|---|---|---|
| | (n = 44) | | (n = 36) | | value |
| Sex, female | 27 | (61) | 30 | (83) | 0.03 |
| Age, years | 60 ± 16 | | 40 ± 16 | | <0.01 |
| Event (death or | 22 | (50) | 4 | (11) | <0.01 |
| LT) | | | | | |
| NYHA I-II/III/IV | 14 (32)/25 (57)/5 (11) | | 14 (39)/20 (56)/2 (5) | | 0.59 |
| 6MWD, m | 309 ± 134 | | 383 ± 144 | | 0.02 |
| RAP, mmHg | 10 ± 5 | | 7 ± 4 | | <0.01 |
| mPAP, mmHg | 52 ± 10 | | 49 ± 13 | | 0.24 |
| PAWP, mmHg | 9 ± 3 | | 8 ± 3 | | 0.35 |
| CO, L/min | 4.4 ± 1.2 | | 4.5 ± 1.2 | | 0.56 |
| CI, L/min/m2 | 2.4 ± 0.6 | | 2.6 ± 0.7 | | 0.19 |
| PVR, WU | 11 ± 4 | | 10 ± 5 | | 0.47 |
| SvO₂, % | 62 ± 9 | | 64 ± 7 | | 0.45 |
| BNP, ng/L | 202 | (83-558) | 71 | (38-245) | 0.01 |
| GDF15, pg/mL | 3047 | (1781-4311) | 818 | (601-1120) | <0.01 |
| Activin B, pg/mL | 122 | (104-171) | 107 | (71-131) | 0.04 |
| FST, pg/mL | 2876 | (2193-4026) | 1957 | (1412-3028) | <0.01 |
| FSTL3, ng/mL | 19 | (15-20) | 12 | (10-15) | <0.01 |
| Inhibin-α | 34 | (4-87) | 47 | (8-77) | 0.33 |
| subunit, pg/mL | | | | | |
| β-NGF, pg/mL | 4.2 | (3.5-5.2) | 3.4 | (2.5-3.8) | 0.01 |
| CXCL9, pg/mL | 787 | (554-1175) | 449 | (349-679) | <0.01 |
| IL-1α, pg/mL | 0.15 | (0.04-0.29) | 0.14 | (0.09-0.25) | 0.83 |
| IL-6, pg/mL | 5.1 | (3.5-7.8) | 2.7 | (1.7-4.0) | <0.01 |

Abbreviations: 6MWD: 6-minute walk distance; β-NGF: beta-nerve growth factor; BNP; brain natriuretic peptide; CI: cardiac index; CO: cardiac output, CXCL: chemokine (C-X-C motif) ligand; FST: Follistatin; FSTL3: Follistatin-like 3; IL: interleukin; mPAP: mean pulmonary artery pressure; NYHA: New York heart association; PAH: pulmonary arterial hypertension; PAWP: pulmonary artery wedge pressure; PVR: pulmonary vascular resistance; RAP: right atrial pressure; Sv02: mixed venous oxygen saturation.

TABLE 4
Multivariable Cox regression analysis including the 3 non-invasive low risk
variables and activin-A (Model A) or FSTL3 (Model B) assessed at diagnosis
and follow-up in discovery EFORT cohort. (Thresholds determined by time-
dependent ROC curves: 393 pg/mL for Activin-A and 16.6 ng/mL for FSTL3).

| Model A | | | Model B | | |
|---|---|---|---|---|---|
| Hazard- | | p- | Hazard- | | |
| ratios | _{95%}CI | value | ratios | _{95%}CI | p-value |

At diagnosis:

| | | | | | | |
|---|---|---|---|---|---|---|
| NYHA | 1.38 | 0.58-3.30 | 0.471 | 1.23 | 0.50-2.99 | 0.653 |
| functional class | | | | | | |
| I-II | | | | | | |
| 6MWD > 440 m | 1.15 | 0.26-5.19 | 0.853 | 0.99 | 0.26-3.75 | 0.993 |
| BNP < 50 or | 0.27 | 0.05-1.59 | 0.147 | 0.30 | 0.06-1.49 | 0.140 |
| NT-proBNP < | | | | | | |
| 300 ng/L | | | | | | |
| Activin A < | 0.14 | 0.03-0.61 | 0.009 | | | |
| threshold | | | | | | |
| FSTL3 < | | | | 0.17 | 0.06-0.45 | <0.001 |
| threshold | | | | | | |

At first follow-up:

| | | | | | | |
|---|---|---|---|---|---|---|
| NYHA | 0.50 | 0.21-1.15 | 0.103 | 0.68 | 0.29-1.57 | 0.362 |
| functional class | | | | | | |
| I-II | | | | | | |
| 6MWD > 440 m | 1.06 | 0.28-4.04 | 0.932 | 0.55 | 0.16-1.87 | 0.336 |
| BNP < 50 or | 0.38 | 0.12-1.27 | 0.116 | 0.47 | 0.14-1.57 | 0.218 |
| NT-proBNP < | | | | | | |
| 300 ng/L | | | | | | |
| Activin A < | 0.23 | 0.07-0.78 | 0.019 | | | |
| threshold | | | | | | |
| FSTL3 < | | | | 0.27 | 0.09-0.78 | 0.015 |
| threshold | | | | | | |

TABLE 5
Antibodies

| | | | Working |
|---|---|---|---|
| Target antigen | Vendor | Catalog # | concentration |
| α-smooth muscle actin | Santa Cruz | Sc-32251 | 1:200 |
| (α-SMA) | Biotechnology | | |
| | Novus | NB300-978 | 1:200 |
| ActRIIA | Santa Cruz | sc-515826 | 1:50 |
| | Biotechnology | | |
| ActrRIIB | Santa Cruz | sc-376593 | 1:50 |
| | Biotechnology | | |
| ALK2 | Abcam | ab155981 | 1:100 |
| ALK4 | Abcam | ab109300 | 1:100 |
| ALK5 | R&D | AF3025 | 1:40 |
| ALK7 | R&D | MAB7749 | 1:40 |
| BAMBI | R&D | AF921 | 1:100 |
| Betaglycan (TGFβRIII) | R&D | AF-242-PB | 1:100 |
| CD31 | R&D | AF3628 | 1:50 |
| Crypto | R&D | MAB2772 | 1:100 |
| CTGF | Cell signaling | #86641 | 1:50 |
| inhibin-α | Abcam | ab47720 | 1:200 |
| inhibin-βA | R&D | AF338 | 1:100 |
| inhibin-βB | R&D | MAB659 | 1:100 |
| Follistatin | R&D | AF669 | 1:100 |
| FTSL3 | R&D | AF1288 | 1:200 |
| PAI-1 | R&D | AF1786 | 1:40 |
| von Willebrand factor | Dako | A0082 | 1:200 |
| (vWF) | | | |

TABLE 6
Comparisons of biomarkers of activin pathway in event-free
survivors and non-survivors, at baseline and follow-up

| | | |
|---|---|---|
| Event (death or lung | Alive without | |
| transplantation) | transplantation | P- |
| (n = 26) | (n = 54) | value |

At baseline

| | | | | | |
|---|---|---|---|---|---|
| Activin A, pg/mL | 633 | (483-852) | 403 | (278-595) | 0.001 |
| Activin B, pg/mL | 114 | (91-142) | 113 | (84-149) | 0.899 |
| Follistatin, pg/mL | 2971 | (2436-3451) | 2182 | (1576-3054) | 0.007 |
| FSTL3, ng/ml | 20 | (18-21) | 14 | (11-17) | <0.001 |
| Inhibin-α subunit, | 16 | (4-78) | 48 | (6-85) | 0.317 |
| pg/ml | | | | | |

At first follow-up

| | | | | | |
|---|---|---|---|---|---|
| Activin A, pg/mL | 655 | (438-872) | 358 | (294-523) | <0.001 |
| Activin B, pg/mL | 86 | (61-125) | 96 | (76-121) | 0.517 |
| Follistatin, pg/mL | 2826 | (2265-3360) | 2118 | (1530-2638) | 0.001 |
| FSTL3, ng/ml | 20 | (17-20) | 14 | (11-16) | <0.001 |
| Inhibin-α subunit, | 28 | (3-75) | 64 | (10-100) | 0.177 |
| pg/mL | | | | | |

Event defined as death or lung transplantation. Data are expressed as median (interquartile range 25%-75%).

TABLE 7
Baseline characteristics for PAH patients in the validation cohort

| | Incident PAH patients from | |
|---|---|---|
| | validation cohort (n = 125) | |
| Female, n (%) | 86 | (69) |
| Age, years | 59 ± 17 | |
| PAH etiology, n (%) | | |
| Idiopathic | 114 | (91) |
| Heritable | 11 | (9) |
| Drug-induced PAH | 0 | |
| NYHA functional class, n (%) | | |
| I-II/III/IV | 10 (8)/94 (75)/21(17) | |
| 6MWD, m | 230 ± 160 | |
| RHC | | |
| RAP, mmHg | 10 ± 5 | |
| mPAP, mmHg | 50 ± 12 | |
| PAWP, mmHg | 9 ± 3 | |
| CO, L/min | 3.8 ± 1.4 | |
| CI, L/min/m² | 2.1 ± 0.8 | |
| PVR, WU | 12 ± 6 | |
| SvO₂, % | 64 ± 11 | |
| BNP, ng/L (EFORT cohort n = 72) | 274 | (81-544) |
| NTproBNP, ng/L (EFORT cohort n = 4) | NA | |
| First line treatment, n (%) | | |
| Calcium channel blocker | 6 | (5) |
| Oral monotherapy | 75 | (60) |
| Oral dual combination therapy | 35 | (28) |
| Dual combination therapy including IV | 6 | (5) |
| prostacyclin | | |
| Triple combination therapy | 3 | (2) |

### Example 2 Levels of Activin-A and FSTL3 Predict Microvasculopathy Extension, Residual PH and Survival in Subject Eligible for PEA

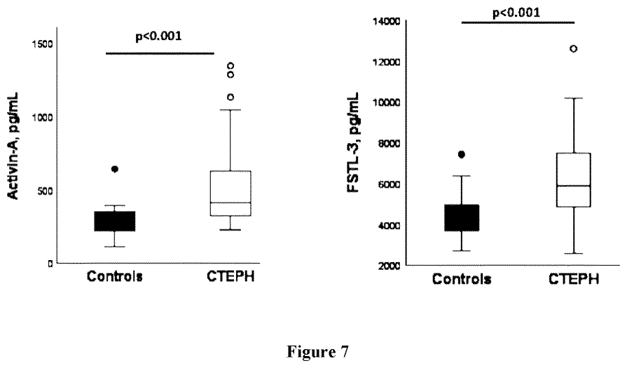

First, we measured the circulating levels of Activin-A and FSTL3 in operable CTEPH patients compared to healthy controls. Then, we investigated if these biomarkers are predictors of responsiveness to PEA (defined by mean pulmonary arterial pressure (mPAP)≥25 mmHg at least 3 months post-surgery). Plasma samples were obtained from CTEPH patients eligible for PEA shortly before surgery. Clinical and hemodynamical data were collected at the time of CTEPH diagnosis, before surgery and at least 3 months after surgery. FSTL-3 and activin-A biomarkers were measured using standard ELISA. A total of 72 CTEPH (mean age 62±13 years; 53% males) and 43 controls (mean age 44±123 years; 65% males) were enrolled. Before PEA, NYHA-functional class was III or IV in 40% of patients, with mean pulmonary arterial pressure (mPAP) of 40±11 mmHg, mean cardiac index of 2.7±0.6 L/min/m2, and pulmonary vascular resistance (PVR) of 6.0±3.7 WU. At the time of PEA, 26% of patients received specific pulmonary vasodilators. Levels activin-A and FSTL-3 were significantly higher in CTEPH patients compared to controls (p<0.001 in all cases) (FIG. 1). Out of 68 patients presenting a follow-up assessment at least 3 months post-PEA, higher levels of mPAP, (OR 1.114, CI95% 1.033-1.202, p=0.005), FSTL-3 (OR 1.000, CI95% 1.000-1.0001; p=0.031) and activin-A (OR 1.003, CI95% 1.001-1.006; p=0.003) before surgery were predictors of mPAP>25 mmHg at least 3 months after surgery, using logistic regression (table 1). The relationship between elevated serum levels of activin A or FSTL3 and unresponsiveneness to PEA persisted in a model adjusted for mPAP (FIG. 7 & Table 8).

These novel biomarkers may aid in the initial assessment of CTEPH patients eligible for PEA. Notably, CTEPH patients exhibit higher plasma concentrations of activin-A and FSTL-3 compared to controls. Furthermore, higher concentration of Activin A and FSTL-3 before surgery are predictive of worse surgical outcome, defined by mPAP≥25 mmHG at least 3 months after PEA.

TABLE 8
Baseline characteristics for PAH patients in the validation cohort

| Variables | OR | 95% CI | p-value |
|---|---|---|---|
| Activin A, pg/mL | 1.003 | 1.001-1.005 | 0.003 |
| FSTL-3, pg/mL | 1.000 | 1.000-1.001 | 0.014 |
| mPAP, mmHg | 1.114 | 1.033-1.202 | 0.005 |
| PCWP, mmHg | 1.177 | 0.967-1.433 | 0.105 |
| RAP, mmHG | 1.131 | 0.965-1.326 | 0.127 |
| PVR, WU | 1.166 | 0.967-1.406 | 0.108 |
| CI, L/min/m² | 1.413 | 0.610-3.269 | 0.420 |
| SvO2, % | 0.965 | 0.893-1.042 | 0.362 |
| 6MWD, m | 0.998 | 0.993-1.002 | 0.265 |
| NTproBNP, pg/mL | 1.000 | 1.000-1.001 | 0.178 |

### REFERENCES

Throughout this application, various references describe the state of the art to which this invention pertains. The disclosures of these references are hereby incorporated by reference into the present disclosure.
1. Simonneau G, Montani D, Celermajer D S, Denton C P, Gatzoulis M A, Krowka M, Williams P G and Souza R. Haemodynamic definitions and updated clinical classification of pulmonary hypertension. Eur Respir J. 2019; 53.
2. Humbert M, Guignabert C, Bonnet S, Dorfmuller P, Klinger J R, Nicolls M R, Olschewski A J, Pullamsetti S S, Schermuly R T, Stenmark K R and Rabinovitch M. Pathology and pathobiology of pulmonary hypertension: state of the art and research perspectives. Eur Respir J. 2019; 53.
3. Humbert M, Sitbon O, Chaouat A, Bertocchi M, Habib G, Gressin V, Yaici A, Weitzenblum E, Cordier J F, Chabot F, Dromer C, Pison C, Reynaud-Gaubert M, Haloun A, Laurent M, Hachulla E, Cottin V, Degano B, Jais X, Montani D, Souza R and Simonneau G. Survival in patients with idiopathic, familial, and anorexigen-associated pulmonary arterial hypertension in the modern management era. Circulation. 2010; 122:156-63.
4. Farber H W, Miller D P, Poms A D, Badesch D B, Frost A E, Muros-Le Rouzic E, Romero A J, Benton W W, Elliott C G, McGoon M D and Benza R L. Five-Year outcomes of patients enrolled in the REVEAL Registry. Chest. 2015; 148:1043-54.
5. Boucly A, Cottin V, Nunes H, Jais X, Tazi A, Prevot G, Reynaud-Gaubert M, Dromer C, Viacroze C, Horeau-Langlard D, Pison C, Bergot E, Traclet J, Weatherald J, Simonneau G, Valeyre D, Montani D, Humbert M, Sitbon O and Savale L. Management and long-term outcomes of sarcoidosis-associated pulmonary hypertension. Eur Respir J. 2017; 50.
6. Savale L, Le Pavec J, Mercier O, Mussot S, Jais X, Fabre D, O'Connell C, Montani D, Stephan F, Sitbon O, Simonneau G, Dartevelle P, Humbert M and Fadel E. Impact of High-Priority Allocation on Lung and Heart-Lung Transplantation for Pulmonary Hypertension. Ann Thorac Surg. 2017; 104:404-411.
7. Aldred M A, Morrell N W and Guignabert C. New Mutations and Pathogenesis of Pulmonary Hypertension: Progress and Puzzles in Disease Pathogenesis. Circulation research. 2022; 130:1365-1381.
8. Guignabert C and Humbert M. Targeting transforming growth factor-beta receptors in pulmonary hypertension. Eur Respir J. 2021; 57.
9 Ricard N, Tu L, Le Hiress M, Huertas A, Phan C, Thuillet R, Sattler C, Fadel E, Seferian A, Montani D, Dorfmuller P, Humbert M and Guignabert C. Increased pericyte coverage mediated by endothelial-derived fibroblast growth factor-2 and interleukin-6 is a source of smooth muscle-like cells in pulmonary hypertension. Circulation. 2014; 129: 1586-97.
10. Yung L M, Nikolic I, Paskin-Flerlage S D, Pearsall R S, Kumar R and Yu P B. A Selective Transforming Growth Factor-beta Ligand Trap Attenuates Pulmonary Hypertension. Am J Respir Crit Care Med. 2016; 194:1140-1151.
11. Yung L M, Yang P, Joshi S, Augur Z M, Kim S S J, Bocobo G A, Dinter T, Troncone L, Chen P S, McNeil M E, Southwood M, Poli de Frias S, Knopf J, Rosas I O, Sako D, Pearsall R S, Quisel J D, Li G, Kumar R and Yu P B. ACTRIIA-Fc rebalances activin/GDF versus BMP signaling in pulmonary hypertension. Sci Transl Med. 2020; 12.
12. Humbert M, McLaughlin V, Gibbs J S R, Gomberg-Maitland M, Hoeper M M, Preston I R, Souza R, Waxman A, Escribano Subias P, Feldman J, Meyer G, Montani D, Olsson K M, Manimaran S, Barnes J, Linde P G, de Oliveira Pena J, Badesch D B and Investigators P T. Sotatercept for the Treatment of Pulmonary Arterial Hypertension. N Engl J Med. 2021; 384:1204-1215.
13. Ryanto G R T, Ikeda K, Miyagawa K, Tu L, Guignabert C, Humbert M, Fujiyama T, Yanagisawa M, Hirata K I and Emoto N. An endothelial activin A-bone morphogenetic protein receptor type 2 link is overdriven in pulmonary hypertension. Nat Commun. 2021; 12:1720.
14. Yndestad A, Larsen K O, Oie E, Ueland T, Smith C, Halvorsen B, Sjaastad I, Skjonsberg O H, Pedersen T M, Anfinsen O G, Damas J K, Christensen G, Aukrust P and Andreassen A K. Elevated levels of activin A in clinical and experimental pulmonary hypertension. J Appl Physiol (1985). 2009; 106:1356-64.
15. Fernandez-Ruiz I. Sotatercept therapy for PAH. Nat Rev Cardiol. 2021; 18:386.
16. Samitas K, Poulos N, Semitekolou M, Morianos I, Tousa S, Economidou E, Robinson D S, Kariyawasam H H, Zervas E, Corrigan C J, Ying S, Xanthou G and Gaga M. Activin-A is overexpressed in severe asthma and is implicated in angiogenic processes. Eur Respir J. 2016; 47:769-82.
17. Ueland T, Aukrust P, Aakhus S, Smith C, Endresen K, Birkeland K I, Gullestad L and Johansen O E. Activin A and cardiovascular disease in type 2 diabetes mellitus. Diab Vasc Dis Res. 2012; 9:234-7.
18. Engelse M A, Neele J M, van Achterberg T A, van Aken B E, van Schaik R H, Pannekoek H and de Vries C J. Human activin-A is expressed in the atherosclerotic lesion and promotes the contractile phenotype of smooth muscle cells. Circulation research. 1999; 85:931-9.
19. Bartholin L, Maguer-Satta V, Hayette S, Martel S, Gadoux M, Corbo L, Magaud J P and Rimokh R. Transcription activation of FLRG and follistatin by activin A, through Smad proteins, participates in a negative feedback loop to modulate activin A function. Oncogene. 2002; 21:2227-35.
20. Sitbon O, Clerson P, Cottin V, Degroote P, Prevot G, Savale L, Jaïs X, Chaouat A, Dromer C, Bourdin A, Renard S, Bergot E, Pison C, Bauer F, Dauphin C, Reynaud Gaubert M, Montani D, Simonneau G and Humbert M. A dynamic prognostic model to predict survival and determine treatment goals in pulmonary arterial hypertension (PAH): the EFORT study. European Respiratory Journal. 2019; 54:OA497.
21. Huertas A, Tu L, Humbert M and Guignabert C. Chronic inflammation within the vascular wall in pulmonary arterial hypertension: more than a spectator. Cardiovasc Res. 2020; 116:885-893.
22. Sweatt A J, Hedlin H K, Balasubramanian V, Hsi A, Blum L K, Robinson W H, Haddad F, Hickey P M, Condliffe R, Lawrie A, Nicolls M R, Rabinovitch M, Khatri P and Zamanian R T. Discovery of Distinct Immune Phenotypes Using Machine Learning in Pulmonary Arterial Hypertension. Circulation research. 2019; 124:904-919.
23. Nakamura T, Takio K, Eto Y, Shibai H, Titani K and Sugino H. Activin-binding protein from rat ovary is follistatin. Science. 1990; 247:836-8.
24. Sugino K, Kurosawa N, Nakamura T, Takio K, Shimasaki S, Ling N, Titani K and Sugino H. Molecular heterogeneity of follistatin, an activin-binding protein. Higher affinity of the carboxyl-terminal truncated forms for heparan sulfate proteoglycans on the ovarian granulosa cell. J Biol Chem. 1993; 268:15579-87.
25. Lewis K A, Gray P C, Blount A L, MacConell L A, Wiater E, Bilezikjian L M and Vale W. Betaglycan binds inhibin and can mediate functional antagonism of activin signalling. Nature. 2000; 404:411-4.
26. Onichtchouk D, Chen Y G, Dosch R, Gawantka V, Delius H, Massague J and Niehrs C. Silencing of TGF-beta signalling by the pseudoreceptor BAMBI. Nature. 1999; 401:480-5.
27. Kelber J A, Shani G, Booker E C, Vale W W and Gray P C. Cripto is a noncompetitive activin antagonist that forms analogous signaling complexes with activin and nodal. J Biol Chem. 2008; 283:4490-500.
28. Galie N, Humbert M, Vachiery J L, Gibbs S, Lang I, Torbicki A, Simonneau G, Peacock A, Vonk Noordegraaf A, Beghetti M, Ghofrani A, Gomez Sanchez M A, Hansmann G, Klepetko W, Lancellotti P, Matucci M, McDonagh T, Pierard L A, Trindade P T, Zompatori M and Hoeper M. 2015 ESC/ERS Guidelines for the diagnosis and treatment of pulmonary hypertension: The Joint Task Force for the Diagnosis and Treatment of Pulmonary Hypertension of the European Society of Cardiology (ESC) and the European Respiratory Society (ERS): Endorsed by: Association for European Paediatric and Congenital Cardiology (AEPC), International Society for Heart and Lung Transplantation (ISHLT). Eur Respir J. 2015; 46:903-75.
29. Boucly A, Weatherald J, Savale L, Jais X, Cottin V, Prevot G, Picard F, de Groote P, Jevnikar M, Bergot E, Chaouat A, Chabanne C, Bourdin A, Parent F, Montani D, Simonneau G, Humbert M and Sitbon O. Risk assessment, prognosis and guideline implementation in pulmonary arterial hypertension. Eur Respir J. 2017; 50.
30. Boucly A, Weatherald J, Savale L, de Groote P, Cottin V, Prevot G, Chaouat A, Picard F, Horeau-Langlard D, Bourdin A, Jutant E M, Beurnier A, Jevnikar M, Jais X, Simonneau G, Montani D, Sitbon O and Humbert M. External validation of a refined 4-strata risk assessment score from the French pulmonary hypertension Registry. Eur Respir J. 2021.
31. Benza R L, Gomberg-Maitland M, Elliott C G, Farber H W, Foreman A J, Frost A E, McGoon M D, Pasta D J, Selej M, Burger C D and Frantz R P. Predicting Survival in Patients With Pulmonary Arterial Hypertension: The REVEAL Risk Score Calculator 2.0 and Comparison With ESC/ERS-Based Risk Assessment Strategies. Chest. 2019; 156:323-337.
32. Kylhammar D, Kjellstrom B, Hjalmarsson C, Jansson K, Nisell M, Soderberg S, Wikstrom G and Radegran G. A comprehensive risk stratification at early follow-up determines prognosis in pulmonary arterial hypertension. Eur Heart J. 2018; 39:4175-4181.
33. Harbaum L, Rhodes C J, Wharton J, Lawrie A, Karnes J H, Desai A A, Nichols W C, Humbert M, Montani D, Girerd B, Sitbon O, Boehm M, Novoyatleva T, Schermuly R T, Ghofrani A, Toshner M, Kiely D G, Howard L S, Swietlik E M, Graf S, Pietzner M, Morrell N W and Wilkins M R. Mining the Plasma Proteome for Insights into the Molecular Pathology of Pulmonary Arterial Hypertension. Am J Respir Crit Care Med. 2022.
34. Rhodes C J, Wharton J, Swietlik E M, Harbaum L, Girerd B, Coghlan J G, Lordan J, Church C, Pepke-Zaba J, Toshner M, Wort S J, Kiely D G, Condliffe R, Lawrie A, Graf S, Montani D, Boucly A, Sitbon O, Humbert M, Howard L S, Morrell N W and Wilkins M R. Using the Plasma Proteome for Risk Stratifying Patients with Pulmonary Arterial Hypertension. Am J Respir Crit Care Med. 2022; 205:1102-1111.
35. Kariotis S, Jammeh E, Swietlik E M, Pickworth J A, Rhodes C J, Otero P, Wharton J, Iremonger J, Dunning M J, Pandya D, Mascarenhas T S, Errington N, Thompson A A R, Romanoski C E, Rischard F, Garcia J G N, Yuan J X, An T S, Desai A A, Coghlan G, Lordan J, Corris P A, Howard L S, Condliffe R, Kiely D G, Church C, Pepke-Zaba J, Toshner M, Wort S, Graf S, Morrell N W, Wilkins M R, Lawrie A, Wang D and Consortium UKNPCS. Biological heterogeneity in idiopathic pulmonary arterial hypertension identified through unsupervised transcriptomic profiling of whole blood. Nat Commun. 2021; 12:7104.
36. Cracowski J L, Chabot F, Labarere J, Faure P, Degano B, Schwebel C, Chaouat A, Reynaud-Gaubert M, Cracowski C, Sitbon O, Yaici A, Simonneau G and Humbert M. Proinflammatory cytokine levels are linked to death in pulmonary arterial hypertension. Eur Respir J. 2014; 43:915-7.
37. Tamura Y, Phan C, Tu L, Le Hiress M, Thuillet R, Jutant E M, Fadel E, Savale L, Huertas A, Humbert M and Guignabert C. Ectopic upregulation of membrane-bound IL6R drives vascular remodeling in pulmonary arterial hypertension. The Journal of clinical investigation. 2018; 128:1956-1970.
38. Hernandez-Sanchez J, Harlow L, Church C, Gaine S, Knightbridge E, Bunclark K, Gor D, Bedding A, Morrell N, Corris P and Toshner M. Clinical trial protocol for TRANSFORM-UK:

A therapeutic open-label study of tocilizumab in the treatment of pulmonary arterial hypertension. Pulm Circ. 2018; 8:2045893217735820.
39. Spiekerkoetter E, Sung Y K, Sudheendra D, Scott V, Del Rosario P, Bill M, Haddad F, Long-Boyle J, Hedlin H and Zamanian R T. Randomised placebo-controlled safety and tolerability trial of FK506 (tacrolimus) for pulmonary arterial hypertension. Eur Respir J. 2017; 50.
40. Montani D, Souza R, Binkert C, Fischli W, Simonneau G, Clozel M and Humbert M. Endothelin-1/endothelin-3 ratio: a potential prognostic factor of pulmonary arterial hypertension. Chest. 2007; 131:101-8.
41. Joshi S R, Liu J, Bloom T, Karaca Atabay E, Kuo T H, Lee M, Belcheva E, Spaits M, Grenha R, Maguire M C, Frost J L, Wang K, Briscoe S D, Alexander M J, Herrin B R, Castonguay R, Pearsall R S, Andre P, Yu P B, Kumar R and Li G. Sotatercept analog suppresses inflammation to reverse experimental pulmonary arterial hypertension. Sci Rep. 2022; 12:7803.

1. An in vitro method for assessing a subject's risk of having or developing a severe form of Pulmonary Hypertension (PH) and then treating the subject, comprising
i) determining in a sample obtained from the subject a level of follistatin-like 3 (FSTL3) alone or optionally with a level of Activin A
ii) determining that the level of FSTL3 is higher than a reference value for FSTL3 or that the levels of both FSTL3 and Activin A are higher than reference values for each of FSTL3 and Activin A, and
iii) treating the subject determined to have a high level of FSTL3 or high levels of both FSTL3 and Activin A with a suitable PH treatment.

2. The in vitro method according to claim 1, wherein the sample is a blood sample.

3. An in-vitro method for assessing a Pulmonary Hypertension (PH) patient's risk of having a poor prognostic of survival and then treating the patient, comprising
i) determining in a sample obtained from the subject a level of follistatin-like 3 (FSTL3) alone or optionally with a level Activin A
ii) determining that the level of FSTL3 is higher than a reference value for FSTL3 or that the levels of both FSTL3 and Activin A are higher than reference values for each of FSTL3 and Activin A, and
iii) treating the subject determined to have a high level of FSTL3 or high levels of both FSTL3 and Activin A with a heart-lung transplant.

4. An in vitro method for monitoring Pulmonary Hypertension (PH) disease in a subject in need thereof and then treating the subject comprising
i) determining a level of follistatin-like 3 (FSTL3) alone or optionally with a level of Activin A in a sample obtained from the subject at a first specific time of the PH disease,
ii) determining the level of follistatin-like 3 (FSTL3) alone or optionally with the level of Activin A in a sample obtained from the subject at a second specific time of the PH disease,
iii) treating the subject determined to have an increased level of FSTL3 or increased levels of both FSTL3 and Activin A at the second specific time of the PH disease with a suitable PH treatment.

5. An in vitro method for monitoring a treatment of Pulmonary Hypertension (PH) in a subject comprising the steps of
i) determining a level of follistatin-like 3 (FSTL3) alone or optionally with a level of Activin A in a sample obtained from the subject before the treatment,
ii) determining the level of follistatin-like 3 (FSTL3) alone or optionally with the level of Activin A in a sample obtained from the subject after the treatment,
iii) determining that the treatment is efficient when the level of follistatin-like 3 (FSTL3) alone or optionally with the level of Activin A determined at step ii) is/are lower than the level(s) determined at step i), and
iv) treating the subject determined to have a lower level of FSTL3 or lower levels of both FSTL3 and Activin A after the treatment with a suitable treatment.

6. The in vitro method according to claim 3, wherein the sample is a blood sample.

7. The in vitro method according to claim 1, wherein the Pulmonary Hypertension (PH) is Pulmonary Arterial Hypertension (PAH).

8. The in vitro method according to claim 7, wherein the Pulmonary Arterial Hypertension (PAH) is selected from the list group consisting of: idiopathic Pulmonary Arterial Hypertension (iPAH), heritable Pulmonary Arterial Hypertension (heritable PAH) and drug- and toxin-induced (anorexigen) Pulmonary Arterial Hypertension (toxin-induced PAH).

9. (canceled)

10. A method of treating Pulmonary Hypertension (PH) in a subject comprising the steps of:
a) providing a blood sample from the subject,
b) detecting a level of follistatin-like 3 (FSTL3) alone or optionally with a level of Activin A in the blood sample,
c) determining that the level of FSTL3 is lower than a reference value for FSTL3 or that the levels of both FSTL3 and Activin A are lower than reference values for each of FSTL3 and Activin A and treating the subject determined to have a lower level of FSTL3 or lower levels of both FSTL3 and Activin A with endothelin receptor antagonists and/or phosphodiesterase type 5 (PDE-5) inhibitors and/or prostacyclin derivatives or
determining that the level of FSTL3 is higher than a reference value for FSTL3 or that the levels of both FSTL3 and Activin A are higher than reference values for each of FSTL3 and Activin A and treating the subject determined to have a higher level of FSTL3 or higher levels of both FSTL3 and Activin A with an escalated treatment with endothelin receptor antagonists and/or phosphodiesterase type 5 (PDE-5) inhibitors and/or prostacyclin derivatives and/or lung transplantation.

11. The method according to claim 10, wherein the Pulmonary Hypertension (PH) is Pulmonary Arterial Hypertension (PAH).

12. The method according to claim 11, wherein Pulmonary Arterial Hypertension (PAH) is selected from the group consisting of: idiopathic Pulmonary Arterial Hypertension (iPAH), heritable Pulmonary Arterial Hypertension (heritable PAH) and drug- and toxin-induced (anorexigen) Pulmonary Arterial Hypertension (toxin-induced PAH).

13. The in vitro method according to claim 1, wherein the suitable PH treatment is i) administration of at least one of: an endothelin receptor antagonist (ERA), a phosphodiesterase type-5 inhibitor and a prostacyclin analog therapy; or ii) lung surgery.
